SEQUENCE LISTING (1 sequence)

SEQ ID NO: 1 - protein, 12 aa, Artificial Seuqence
  Synthetic
GTLVTVSSASTK

(19) United States
(12) Patent Application Publication
(10) Pub. No.: US 2023/0176068 A1
(43) Pub. Date: Jun. 8, 2023

(54) MEASUREMENT OF AFUCOSYLATED IGG FC GLYCANS AND RELATED COVID-19 TREATMENT METHODS

(71) Applicants: CHAN ZUCKERBERS BIOHUB, INC., San Francisco, CA (US); THE BOARD OF TRUSTEES OF THE LELAND STANFORD JUNIOR UNIVERSITY, Stanford, CA (US)

(72) Inventor: Taia Wang, Main Quad, CA (US)

(21) Appl. No.: 17/923,672

(22) PCT Filed: May 10, 2021
(86) PCT No.: PCT/US2021/031591

§ 371 Date: Nov. 7, 2022

Related U.S. Application Data
(60) Provisional application No. 63/088,316, filed on Oct. 6, 2020.
(60) Provisional application No. 63/023,079, filed on May 11, 2020.

(51) Int. Cl. G01N 33/68; G01N 33/569

(52) U.S. Cl. CPC G01N 33/6854; G01N 33/56983; G01N 2440/38; G01N 2469/20

(57) ABSTRACT

The present disclosure provides materials and methods for identifying patients that are at risk of progression to clinically significant COVID-19 infection or disease.

### CROSS REFERENCE TO RELATED APPLICATIONS

The present application claims priority to U.S. Provisional Pat. Application No. 63/023,079, filed May 11, 2020 and U.S. Provisional Pat. Application No. 63/088,316, filed Oct. 6, 2020 the entirety of each of which are incorporated by reference herein.

### STATEMENT REGARDING FEDERALLY SPONSORED RESEARCH

This invention was made with Government support under contract AI111825 awarded by the National Institutes of Health. The Government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55623_Seqlisting.txt”, which was created on May 10, 2021 and is 628 bytes in size. The subject matter of the Sequence Listing is incorporated herein in its entirety by reference.

### BACKGROUND

Antibodies formed early during infection can bind to virus particles, forming immune complexes that may neutralize or mediate clearance of virus, but immune complexes can also promote inflammation and exacerbate symptoms of disease via interactions between antibody Fc domains and Fc gamma receptors (FcyRs). How antibodies within immune complexes modulate infections depends, in part, on their Fc domain structure. Antibody structure, in turn, dictates interactions with Fc receptors that are expressed by a variety of cells that can become activated during infectious diseases. Antibody isotypes, IgG, IgA, and IgM are a primary determinant of structure and thus of activity, with IgM production occurring first and signaling onset of new B cell responses. Production of class switched antibodies follows, with IgA central in mucosal immunity and IgG being the dominant isotype in systemic antiviral immunity. IgG effector function is governed by Fc-FcyR interactions which are regulated by IgG subclasses (IgG1, IgG2, IgG3, IgG4) and post-translational modifications of antibodies within immune complexes. Importantly, people produce distinct structural repertoires of IgG Fc domains, with some people producing highly activating repertoires that are characterized by elevated levels of activating IgG subclasses (IgG1 and IgG3) and/or reduced core-fucosylation of the IgG1 Fc domain. In most infections, IgG responses are protective in nature or do not have a significant impact on infection (Bournazos, S., et al., Annu Rev Immunol, 2017. 35: p. 285-311). In rare circumstances however, antibodies with specific Fc structures may cause cell activation or pro-inflammatory activity during infections which can exacerbate symptoms of disease. A striking example of this is dengue virus infections that occur in the presence of reactive, non-neutralizing IgGs. People at highest risk for severe disease during these infections produce antibodies with reduced fucosylation of the Fc; this modification enhances affinity of the Fc for the activating FcyR, FcyRIIIa. Enhanced FcyRIIIa signaling, in turn, modulates dengue disease pathogenesis through multiple pathways (Wang, T.T., et al., Science, 2017. 355(6323): p. 395-398; and Thulin, N.K., Wang T.T., Cell Reports, 2020. IN PRESS.). It remains unknown whether there are any specific features of antibodies produced by patients with COVID-19 disease.

### SUMMARY OF THE INVENTION

In various aspects, the present disclosure provides methods for identifying a subject that is (a) symptomatic or prone to present one or more symptoms of COVID-19 and/or (b) at risk of progression to clinically significant COVID-19 infection or disease, sad method comprising (i) obtaining a biological sample from a subject, (ii) determining the amount of one or more of immunoglobulin fucosylation, galactosylation and/or bisection in the sample, and (iii) comparing the fucosylation, galactosylation and/or bisection from a blood sample from a healthy adult donor; wherein a reduced level of fucosylation, galactosylation and/or bisection when compared with the healthy adult donor is indicative of a subject that is symptomatic or prone to present one or more symptoms of COVID-19 and/or (b) prone to progress to severe COVID-19 disease.

In another embodiment, the immunoglobulin is IgG. In another embodiment, the amount of fucosylation (or afucosylation) is determined. In another embodiment, the amount of fucosylation is determined, wherein the level of afucosylated Fc glycans is defined as 5 percent or greater or 10 percent or greater. In still other embodiments, the subject is a human. In another embodiment, the biological sample is blood or a blood fraction.

The present disclosure also provides, in one embodiment, a method of treating a subject acutely infected with SARS-CoV-2 and at risk of progression to clinically significant COVID-19 infection or disease, the method comprising administering to the subject a therapeutic agent or vaccine following identification the subject is symptomatic or prone to present one or more symptoms of COVID-19 and/or (b) prone to progress to severe COVID-19 disease according to any one of the aforementioned methods.

### BRIEF DESCRIPTION OF THE DRAWINGS

FIG. 1 shows SARS-CoV-2 antibodies in COVID-19 patients and in undiagnosed children. (A) Anti-RBD IgM, IgA, IgG titers in COVID patients who required treatment in the ICU (red) hospitalization, but no ICU (yellow), patients treated on an outpatient basis (light blue), or seropositive children (dark blue) (B) Anti-RBD AUC for the four patient groups are shown. Data shown are representative of at least two experiments performed in triplicate. Violin plots show the distribution of sample values along with median (solid lines) and quartile (broken lines) values. The dashed line indicates the average AUC value of the pre-COVID-19 historical samples. P values in (B) were calculated using one-way ANOVA.

FIG. 2 shows structural properties of anti-RBD IgG Fc domains in adult COVID-19 patients and in seropositive children. (A) The abundance of anti-RBD IgG subclasses was characterized. ICU patients had elevated levels of IgG3 compared to those treated on the floor or outpatients. (B) Anti-RBD IgG1 Fc post-translational modifications were characterized. Patients who were hospitalized (ICU (red) or floor (plum)) had significantly reduced Fc fucosylation (F), when compared with RBD IgGs from outpatients (sky blue) or from children (dark blue). Fc galactosylation (GS0) was significantly higher and sialylation (S) significantly lower in all adult patients compared with children. No significant differences were observed in levels of IgG1 Fc bisection (N). (C) Of the six afucosylated forms quantified, those lacking both core fucose and a bisecting N-acetyl glucosamine (FONO) were substantially enriched in severe COVID-19 patients. Violin plots show the distribution of sample values along with median (solid lines) and quartile (broken lines) values. (D) Cartoon representation of IgG1 Fc glycans and various F0 modifications. P values in (A), (B) and (C) were calculated using one-way ANOVA.

FIG. 3 shows a summary of antibody signatures from COVID-19 patients. Relative multi-dimensional antibody signatures for each group stratified by disease severity are depicted by radar plots. Each feature, isotype (IgM, IgA, IgG) in blue/purple, IgG subclass (IgG1, IgG2, IgG3 and IgG4) in yellow and % abundance of Fc glycoforms (FONO, GS0, S and N) in red/pink, is depicted as a wedge. The size of the wedges indicates the median of the features, normalized to the corresponding outpatient feature.

FIG. 4 shows the regulation of Fc fucosylation (A, B) The level of the afucosylated anti-RBD IgG1 was significantly higher in hospitalized males as compared to hospitalized females in two different cohorts (Orange- Stanford Hospital Center, Blue -Kaiser Permanente Hospitals of Northern California). (C) Significant sex associated differences in afucosylation levels of IgG1 were not present in mild COVID-19 (outpatients). Violin plots in (A), (B) and (C) show the distribution of sample values along with median (solid lines) and quartile (broken lines) values. P values were calculated using Welch’s t test.

FIG. 5 shows FcyRIIIa binding by human anti-RBD IgGs with variable Fc fucosylation. (A) Binding of polyclonal IgGs was determined by biolayer interferometry. The overlay of binding traces for a donor from each group representing varying degree of anti-RBD core afucosylation; low (0-10%), medium (10-20%) and high (>20%) is shown. The kinetic constants were obtained by evaluating the binding at multiple concentrations (3.3 uM followed by 2-fold dilutions) of the analyte as shown (solid circles). The fits are indicated by solid lines. The assay was performed twice and shown are representative traces from one experiment. (B) A strong positive correlation (Pearson correlation coefficient r=0.8643) was observed between the apparent dissociation constant (KD,app) of FcyRIIIa/CD16a and anti-RBD IgG1 core afucosylation in COVID-19 patients as determined by biolayer interferometry. The binding of anti-RBD monoclonal antibody, CR3022 with different core afucosylation levels is also shown (red). (C) Correlation between the level of anti-RBD IgG1 Fc afucosylation and binding to FcyRIIIa. Binding of serum antibodies (n=38) to FcyRIIIa correlated positively with the degree of afucosylation (Pearson correlation coefficient r= 0.6432). Samples were representative of the range of Fc fucosylation over the sample set. (D) Correlation between the level of anti-RBD IgG1 Fc afucosylation and immune complex (IC) mediated NK cell degranulation. The amount of degranulation, measured by fold increase of CD107a+ NK cells over control, correlated positively with the degree of afucosylation of the anti-RBD IgG (Spearman correlation coefficient r= 0.7857). The assay was performed in duplicate with PBMCs from three healthy donors and mean data has been graphed. (E, F) Highly afucosylated immune complexes elicited increased production of inflammatory cytokines IL-6, TNF-a and IL1b. Immune complexes were formed using pooled polyclonal IgGs from COVID patients (E) or recombinant IgG1 mAb 3022 (F) with distinct levels of afucosylation. The assays were performed in duplicate with monocytes from three healthy donors and mean data and standard error of the mean (SEM) has been graphed. P-values between high and low afucosylated immune complexes at each antibody concentration were calculated by paired t-test.

FIG. 6 shows antibody quality and dynamics in a longitudinal cohort of COVID-19 outpatients. (A) Serological analyses were performed on longitudinal samples from a cohort of COVID-19 outpatients (n=120) collected at day 0 (D0, enrollment), day 5 (D5), day 28 (D28), month 7 (M7) and month 10 (M10). (B) SARS-CoV-2 full length spike (S) binding IgG (AUC), half-maximal SARS-CoV-2 pseudovirus neutralizing titers (pNT50) and the ratio of normalized neutralizing to IgG binding titers are shown. (C) The kinetics of neutralizing antibody response over time has been plotted for the outpatient cohort. pNT50 followed two basic patterns over time; in one group (termed as low responder), the pNT50 was below 500 for the duration of the study, while in the other group (termed high responder), pNT50 was greater than 500 but peak neutralizing titers were achieved at two distinct time periods: early after enrollment (D0/D5) (early), or by study day 28 (later). (D) The cross-correlation matrix shows the relationship between multiple features of the antibody response (D0, D5 and D28 pNT50, D0 and D28 IgG and IgA titers) in longitudinally analyzed COVID-19 patient samples. (E) Early high responders in the outpatient cohorts who elicited neutralizing titers within the first 15 days of symptoms had a significantly shorter course of disease (p=0.001). For (B) and (E) the median values have been depicted with a red line and the p-values calculated using Kruskal Wallis test with Dunn’s correction.

FIG. 7 shows that low early neutralizing titers and elevated Fc afucosylation predict disease progression. (A) Anti-RBD IgG1 Fc afucosylation (afuc) was characterized (n=?). (B) Subjects (n=8) within the cohort whose symptoms progressed over time post enrollment requiring an emergency room visit or hospitalization (Progressors) had significantly higher afucosylation than subjects who maintained their asymptomatic or mild status (Non-progressors) (p=0055). (C) The distribution of early neutralizing titers (D0/D5) amongst Progressors (P) and Non-progressors (NP) showed a statistically significant difference (p=0.0374, Fisher’s exact test). (D) The correlation between anti-RBD IgG1 afucosylation and D0 neutralization titers have been plotted. IgG1 afucosylation is inversely correlated with D0 pNT50 (Pearson’s correlation coefficient r=-0.3431, p=0.0376). (E) Mean ROC response and the area under the curve (AUC) with its standard deviation obtained using random forest classifier with 6-fold cross validation has been plotted. (F) PBMCs from subjects isolated on D0 were assessed by flow cytometry for CD16+ monocyte frequencies as a percent of total CD11c+ HLA-DR+ lin- myeloid cells. (G) Quantitative flow cytometry was employed to determine Feγ receptor (FcyR) expression on bulk myeloid cells. (H) The activating to inhibitory ratio (A/I) which was calculated by combining the range normalized, quantitative expression of FcyRs CD16, CD32a and CD32b has been plotted. Progressors had significantly higher A/I as compared to non-progressors (p=0.01). P values in (A-B, F-H) were calculated with unpaired students t test and in (C) with two-sided Fisher’s exact test.

### DETAILED DESCRIPTION

Antibody responses to viral infections in humans are varied and of widely divergent clinical significance. Pre-existing, reactive antibodies or antibodies that are formed early during infection can bind to virus particles, forming immune complexes that may neutralize the virus or mediate clearance of virus. On the other hand, immune complexes can also promote inflammation and exacerbate symptoms of disease. How antibodies within immune complexes modulate infection depends, in part, on their Fc domain structure. Fc structure, in turn, dictates interactions with FcyRs that are expressed by a variety of cells that are activated during infection (Bournazos, S., et al., Annual review of immunology 35, 285-311 (2017)).

Antibody isotypes, IgG, IgA, and IgM are a primary determinant of Fc-structure and thus of activity. Initial B cell responses are characterized by the production of IgM antibodies. Production of class-switched IgA and IgG antibodies follows, IgA playing a central role in mucosal immunity while IgG is the dominant isotype involved in systemic antiviral immunity. IgG functions are governed by interactions between immune complexes and effector immune cells that express FcyRs, the receptors for IgG. The balance of FcyRs that are engaged by immune complexes determines the degree of the inflammatory effector cell response. Activating, low-affinity FcyRs (FcyRIIa and FcyRIIIa) transduce inflammatory signaling through immunoreceptor tyrosine-based activation motifs (ITAMs); in health, ITAM signaling is balanced by immunoreceptor tyrosine-based inhibition motif (ITIM) signaling through the inhibitory FcyR, FcyRIIb (Wang, T.T., Curr Top Microbiol Immunol (2019); and Nimmerjahn, F. & Ravetch, J.V., Advances in immunology 96, 179-204 (2007)) Imbalanced activating to inhibitory FcγR signaling can cause pathologic inflammation leading to disease (Nimmerjahn, F. & Ravetch, J.V., Advances in immunology 96, 179-204 (2007); Nimmerjahn, F. & Ravetch, J.V., Current topics in microbiology and immunology 350, 105-125 (2011); and Clynes, R. et al., The Journal of experimental medicine 189, 179-185 (1999)).

The strength of interactions between immune complexes and various FcyRs is determined by structural diversity within IgG subclasses (IgG1, IgG2, IgG3, IgG4) and post-translational modifications of their Fc domains (Nimmerjahn, F. & Ravetch, J.V., Science 310, 1510-1512 (2005)). Importantly, individuals produce distinct structural repertoires of IgG Fc domains, with some producing highly activating repertoires enriched for features such as IgG1, IgG3 and/or reduced core-fucosylation of the IgG1 Fc domain. Others produce IgG repertoires characterized by higher levels of IgG2 and/or sialylated Fcs that have reduced activating/inflammatory FcγR signaling potential². It remains unknown whether there are any specific features of antibodies produced by mild or severe COVID-19 patients that might modulate antibody signaling to impact the inflammatory response to SARS-CoV-2 virus and/or viral antigens.

The ongoing severe acute respiratory syndrome coronavirus 2 (SARS-CoV-2) pandemic has caused a public health crisis that is exacerbated by our poor understanding of correlates of immunity. SARS-CoV-2 infection can cause Coronavirus Disease 2019 (COVID-19), characterized by pneumonia and cytokine dysregulation, yet SARS-CoV-2 infections can also be mild or asymptomatic (Bai, Y., et al., JAMA, (2020); Hu, Z., et al., Sci China Life Sci, (2020) 63(5): p. 706-711; and Huang, C., et al., Lancet, (2020), 395(10223): p. 497-506). While antibodies have been shown in a variety of in vitro assays to promote coronavirus infections through antibody-dependent enhancement (ADE) mechanisms requiring interactions between IgG antibodies and Fc gamma receptors (FcyRs), the relevance of these observations to coronavirus infections in humans in not known(Yang, Z.Y., et al., Proc Natl Acad Sci USA, 2005, 102(3): p. 797-801; Yip, M.S., et al., Virol J, 2014. 11: p. 82; Jaume, M., et al., J Virol, 2011. 85(20): p. 10582-97; and Wan, Y., et al., J Virol, 2020. 94(5)). In light of ongoing clinical trials examining convalescent serum therapy for COVID-19 patients and expedited SARS-CoV-2 vaccine testing in humans, it is essential to clarify the role of antibodies in the pathogenesis of COVID-19. The present disclosure provides that adults with PCR-diagnosed COVID-19 produce IgG antibodies with a specific Fc domain repertoire that is characterized by reduced fucosylation, a modification that mediates pro-inflammatory interactions with the FcyR, FcyRIIIa. Fc fucosylation was reduced when compared with SARS-CoV-2-seropositive children and relative to adults with symptomatic influenza virus infections. These results demonstrate an antibody correlate of symptomatic SARS-CoV-2 infections in adults and have implications for novel therapeutic strategies targeting FcyRIIIa pathways.

Coronaviruses (CoV) have repeatedly emerged from wildlife hosts into humans and livestock animals to cause epidemics with significant morbidity and mortality. The emergence of SARS-CoV-2, the virus that causes COVID-19 in 2019 and the rapid, global spread of infection in humans highlights the need for developing therapeutics and vaccines to limit coronavirus epidemics (Wu F, et al., 2020, Nature 1-8; Zhou P, et al., 2020, Nature 1-4; and Zhu N, et al., 2020, N Engl J Med NEJMoa2001017).

Coronaviruses (CoVs) are the largest group of viruses belonging to the Nidovirales order, which includes Coronaviridae, Arteriviridae, and Roniviridae families. The Coronavirinae comprise one of two subfamilies in the Coronaviridae family, with the other being the Torovirinae. The Coronavirinae are further subdivided into four groups, the alpha, beta, gamma and delta coronaviruses. The viruses were initially sorted into these groups based on serology but are now divided by phylogenetic clustering.

All viruses in the Nidovirales order are enveloped, non-segmented positive-sense RNA viruses. They all contain very large genomes for RNA viruses, with Coronavirinae having the largest identified RNA genomes, containing approximately 30 kilobase (kb) genomes. Other common features within the Nidovirales order include: i) a highly conserved genomic organization, with a large replicase gene preceding structural and accessory genes; ii) expression of many nonstructural genes by ribosomal frameshifting; iii) several unique or unusual enzymatic activities encoded within the large replicase-transcriptase polyprotein; and iv) expression of downstream genes by synthesis of 3′ nested sub-genomic mRNAs. In fact, the Nidovirales order name is derived from these nested 3′ mRNAs as nido is Latin for “nest”. The major differences within the Nidovirus families are in the number, type, and sizes of the structural proteins. These differences cause significant alterations in the structure and morphology of the nucleocapsids and virions.

Coronaviruses contain a non-segmented, positive-sense RNA genome of ~30 kb. The genome contains a 5′ cap structure along with a 3′ poly (A) tail, allowing it to act as a mRNA for translation of the replicase polyproteins. The replicase gene encoding the nonstructural proteins (Nsps) occupies two-thirds of the genome, about 20 kb, as opposed to the structural and accessory proteins, which make up only about 10 kb of the viral genome. The 5′ end of the genome contains a leader sequence and untranslated region (UTR) that contains multiple stem loop structures required for RNA replication and transcription. Additionally, at the beginning of each structural or accessory gene are transcriptional regulatory sequences (TRSs) that are required for expression of each of these genes (see section on RNA replication). The 3′UTR also contains RNA structures required for replication and synthesis of viral RNA. The organization of the coronavirus genome is 5′-leader-UTR-replicase-S (Spike)-E (Envelope)-M (Membrane)-N (Nucleocapsid)-3′UTR-poly (A) tail with accessory genes interspersed within the structural genes at the 3′ end of the genome. The accessory proteins are almost exclusively non-essential for replication in tissue culture; however some have been shown to have important roles in viral pathogenesis.

The present disclosure provides, in various embodiments, compositions and methods for treating coronaviruses. Non-limiting examples of coronaviruses include SARS-Related coronaviruses, severe acute respiratory syndrome coronavirus-2, (SARS-CoV-2), severe acute respiratory syndrome coronavirus (SARS-CoV), Middle East respiratory syndrome coronavirus (MERS-CoV), human coronavirus 229E (HCoV-229E), human coronavirus 0C43 (HCoV-0C43), human coronavirus HKU1 (HCoV-HKU1), and human coronavirus NL63 (HCoV-NL63).

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are typically classified as either kappa or lambda. Heavy chains are typically classified as gamma, mu, alpha, delta, or epsilon, which in turn define the immunoglobulin classes, IgG, IgM, IgA, IgD and IgE, respectively.

A typical full-length (intact) immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well-characterized fragments that can be produced, inter alia, by digestion with various peptidases. Thus, for example, pepsin digests an antibody below the disulfide linkages in the hinge region to produce F(ab)′₂, a dimer of Fab which itself is a light chain joined to V_{H}-C_{H}I by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W.E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes whole antibodies, antibody fragments either produced by the modification of whole antibodies or synthesized de novo using recombinant DNA methodologies. In certain embodiments antibodies include single chain antibodies (antibodies that exist as a single polypeptide chain), for example, single chain Fv antibodies (scFv) in which a variable heavy and a variable light chain are joined together (directly or through a peptide linker) to form a continuous polypeptide. In certain embodiments the single chain Fv antibody is a covalently linked V_{H}-V_{L} heterodimer that may be expressed from a nucleic acid including V_{H} and V_{L} encoding sequences either joined directly or joined by a peptide-encoding linker (see, e.g., Huston, et al. (1988) Proc. Nat. Acad. Sci. USA, 85: 5879-5883). While the V_{H} and V_{L} are connected to each as a single polypeptide chain, the V_{H} and V_{L} domains associate non-covalently. The first functional antibody molecules to be expressed on the surface of filamentous phage were single-chain Fv’s (scFv), however, alternative expression strategies have also been successful. For example, Fab molecules can be displayed on phage if one of the chains (heavy or light) is fused, for example, to g3 capsid protein and the complementary chain exported to the periplasm as a soluble molecule. The two chains can be encoded on the same or on different replicons. The important point is that the two antibody chains in each Fab molecule assemble post-translationally and the dimer is incorporated into the phage particle via linkage of one of the chains to, e.g., g3p (see, e.g., U.S. Pat. No: 5,733,743). The scFv antibodies and a number of other structures converting the naturally aggregated, but chemically separated light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5, 132,405, and 4,956,778). Accordingly, in certain embodiments, anti-Fc receptor antibodies include, but are not limited to all that have been displayed on phage or yeast (e.g., scFv, Fv, Fab and disulfide linked Fv (see, e.g., Reiter et al. (1995) Protein Eng. 8: 1323-1331)).

The term “monoclonal antibody” refers to an antibody obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are typically highly specific, being directed against a single epitope. In contrast to polyclonal antibody preparations which typically include different antibodies directed against different determinants (epitopes), each monoclonal antibody is directed against a single determinant on the antigen. The term “monoclonal” indicates the character of the antibody as being obtained from, or one of, a substantially homogeneous population of antibodies, and is not to be construed as requiring production of the antibody by any particular method. For example, monoclonal antibodies may be made by a variety of techniques, including, but not limited to, the hybridoma method (see, e.g., Kohler and Milstein. (1975) Nature, 256:495-497; Hongo et al. (1995) Hybridoma, 14 (3): 253-260; Harlow et al. (1988) Antibodies: A Laboratory Manual (Cold Spring Harbor Laboratory Press, 2d ed.); Hammerling et al. (1981) In: Monoclonal Antibodies and T-Cell Hybridomas 563-681 (Elsevier, N.Y.)), recombinant DNA methods (see, e.g., U.S. Pat. No. 4,816,567), phage-display technologies (see, e.g., Clackson et al. (1991) Nature, 352: 624-628; Marks et al. (1992) J. Mol. Biol. 222: 581-597; Sidhu et al. (2004) J. Mol. Biol. 338(2): 299-310; Lee et al. (2004) J. Mol. Biol. 340(5): 1073-1093; and the like), and technologies for producing human or human-like antibodies in animals that have parts or all of the human immunoglobulin loci or genes encoding human immunoglobulin sequences (see, e.g., PCT Patent Publication Nos: WO 1998/24893; WO 1996/34096; WO 1996/33735; and WO 1991/10741; U.S. Pat. Nos: 5,545,807; 5,545,806; 5,569,825; 5,625, 126; 5,633,425; and 5,661,016; Jakobovits et al. (1993) Nature 362: 255-258; Bruggemann et al. (1993) Year in Immunol. 7: 33; Marks et al. (1992) Bio/Technology 10: 779-783; Lonberg et al. (1994) Nature 368: 856-859; Morrison (1994) Nature 368: 812-813; Fishwild et al. (1996) Nature Biotechnol. 14: 845-851); Neuberger (1996) Nature Biotechnol. 14: 826; Lonberg and Fluszar (1995) Intern. Rev. Immunol. 13 : 65-93; and the like).

“Humanized antibodies” are forms of antibodies that contain sequence (typically minimal sequence) derived from non-human (e.g., murine) immunoglobulin the remaining sequence being derived from a human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit or non-human primate having the desired specificity, affinity, and/or capacity. In some instances, framework residues of the human immunoglobulin are replaced by corresponding non-human residues. In certain embodiments. Humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the framework regions are those of a human immunoglobulin sequence, although the framework regions may include one or more individual framework residue substitutions that improve antibody performance, such as binding affinity, isomerization, immunogenicity, and the like. The number of these amino acid substitutions in the framework is typically no more than 6 in the H chain, and in the L chain typically no more than 3. In certain embodiments the humanized antibody can also comprise at least a portion of an immunoglobulin constant region (Fc), typically that of a human immunoglobulin (see, e.g., Jones et al. (1986) Nature 321 : 522-525; Riechmann et al. (1988) Nature 332: 323-329; Presta (1992) Curr. Op. Struct. Biol. 2: 593-596 (1992); Vaswani and Hamilton (1998) Ann. Allergy, Asthma. Immunol. 1 : 105-115; Harris, (1995) Biochem. Soc. Transact. 23 : 1035-1038; Hurle and Gross (1994) Curr. Op. Biotech. 5:428-433; and U.S. Pat. Nos: 6,982,321 and 7,087,409).

The term “Fc region” refers to a C-terminal region of an immunoglobulin heavy chain, including native- sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native- sequence Fc regions for use in the antibodies described herein include, but are not limited to, human IgGl, IgG2, IgG3 and IgG4.

An “Fc receptor” or “FcR” refers to a receptor that typically binds to an Fc region of an antibody. In certain embodiments the FcR is a native sequence human FcR. In certain embodiments the FcR is one that binds an IgG antibody (a gamma receptor) and includes, for example, receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (“ITAM”) in its cytoplasmic domain. Inhibiting receptor FcyRIIB contains an immunoreceptor tyrosine-based inhibition motif (“ITIM”) in its cytoplasmic domain, (see, e.g., Daeron (1997) Annu. Rev. Immunol. 15: 203-234; Ravetch and Kinet (1991) Annu. Rev. Immunol. 9: 457-492 (1991); Capel et al. (1994) Immunometh. 4: 25-34; de Haas et al. (1995) J. Lab. Clin. Med. 126: 330-341). Other FcRs, including those to be identified in the future, are encompassed by the term “FcR” herein.

The terms “binding”, “specific binding”, and “specifically recognizes” are used interchangeably herein and indicates that an antibody exhibits substantial affinity for a specific molecule or fragment(s) thereof and is said to occur when the antibody is selective in that it does not exhibit significant cross-reactivity with other molecules lacking the target epitope. In certain embodiments substantial binding includes binding with a dissociation constant (K_{d}) of 10”⁶, 10”⁷, 10”⁸, 10”⁹, 10”¹⁰, 10”¹¹, 10”¹² M. or better. Values intermediate to those set forth herein are also contemplated, and preferred binding affinity can be indicated as a range of dissociation constants, for example preferred binding affinities for antibodies disclosed herein are represented by K_{d} values ranging from 10”⁶ to 10”¹² M (i.e., micromolar to picomolar), preferably 10” 1 to 10” 12 M, more preferably 10” 1 to 10” 12 M or better. Binding affinity and selectivity can be determined using any art-recognized methods for determining such characteristics, including, for example, using Scatchard analysis and/or competitive (competition) binding assays (see, e.g., Wassaf et al. (2006) Anal. Biochem. 351(2):241-53; Epub 2006 Feb. 10 (BIACORE); and Murray and Brown (1999) J. Immunol. Meth. 127(1): 25-28 (ELISA)).

As used herein, the term “afucosylated Fc glycan” refers to Fc glycans lacking a core fucose on the Fc glycan of the IgG heavy chain.

As used herein, the phrase “determining the amount of one or more of immunoglobulin fucosylation,” “determining the level of afucosylated Fc glycans in IgG antibodies” encompasses determining this level directly, by measuring afucosylated Fc glycans in IgG antibodies, or indirectly, by measuring fucosylated Fc glycans in IgG antibodies. For example, a level of afucosylated Fc glycans in IgG antibodies above 10% can be determined by measuring a level of fucosylated Fc glycans in IgG of less than 90%.

As used herein with reference to infection or disease, the term “clinically significant” refers to infection or disease requiring direct observation and/or treatment by a medical professional.

As used herein, the term “infant” refers to a child during the span of time from birth to 16 months.

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term treat can refer to prophylactic treatment which includes a delay in the onset or the prevention of the onset of a pathology or disease.

As used herein, the term “small molecule” refers to an organic compound having a molecular weight of less than about 900 daltons.

### Antibody Analysis in Coronavirus Infection

As described in International Publication No. WO2019/083904, incorporated by reference in its entirety herein, humans have highly variable Fc domain repertoires, defined as the precise IgG subclass and Fc glycoform distributions of serum IgG. Indeed, the abundance of sialylated and afucosylated glycoforms on serum IgG can vary up to -30% between individuals, while the ratio of the dominant activating to inhibitory IgG subclasses (IgGl/IgG2) varies by -25%. This heterogeneity in determinants of Fc-FcyR binding impacts FcyR signaling and thus modulates vaccine efficacy susceptibility to autoimmune disorders and infectious diseases and likely determines numerous additional antibody-mediated processes.

Afucosylated Fc glycans in IgG antibodies from individuals infected with a coronavirus correlate with progression to clinically significant, and potentially life-threatening disease. For example, afucosylated Fc glycans in IgG antibodies are believed to play a role in progression to severe SARS-CoV-2 disease, i.e., COVID-19.

### Measurement of Afucosylated Fc Glycans

Afucosylated Fc glycans can be measured by any convenient method, including that described in Wang, et al. (supra, which is incorporated by reference for this description). An illustrative method is provided in Example 1 in WO2019/083904, incorporated herein by reference.

In some embodiments, an antibody that distinguishes between fucosylated and afucosylated Fc glycans can be employed in a standard immunoassay.

In some embodiments, the measurement is carried out on IgGl antibodies.

IgG antibodies for the measurement can be obtained from any biological sample reflecting the repertoire of IgG antibodies in the subject, typically blood or a blood fraction.

Afucosylated Fc glycans can be measured in total IgG (i.e., all IgG antibodies in the sample) or in an IgG fraction. For example, IgG subclasses can be isolated using standard techniques, such as that described, for example, in Wang, et al. (supra, which is incorporated by reference for this description) or by Leblebici, et al. (2014), Separation of human immunoglobulin G subclasses on a protein A monolith column, J Chromatogr B Analyt Technol Biomed Life Sci., 962:89-93. The IgG antibodies, whether all subclasses or just IgGl can be IgG antibodies of all specificities. Alternatively, prior to measurement of afucosylated Fc glycans, antigen-specific antibodies can be isolated using any standard technique, such as affinity chromatography. In some embodiments, the IgG antibodies used for the measurement are IgG antibodies specific for a coronavirus antigen.

In various embodiments, afucosylated Fc glycans are considered to be elevated when they make up at least 1, 2, 3, 4, 5, 6, 7, 8, 9, 11, 12, 13, 14, 15, 16, 17, 18, 19, 20, 21, 22, 23, 24, 25, 26, 27, 28, 29, or 30 percent of the IgG antibodies tested. The threshold for elevation is set, in a particular application, based on the desired predictive value of the test.

### Treatment of Coronavirus Infection Based on Antibody Analysis

A subject (including an infant) having at least one symptom of infection with a coronavirus and that has been determined to have an elevated level of afucosylated Fc glycans by assaying a biological sample from the subject is at increased risk for progression to clinically significant coronavirus (COVID-19) infection and/or disease. Likewise, an infant having at least one symptom of infection with a coronavirus, whose mother has been determined to have an elevated level of afucosylated Fc glycans by assaying a biological sample from the mother, is at increased risk for progression to clinically significant coronavirus infection and/or disease. In some embodiments, the subject or the infant and/or mother will have tested positive for a coronavirus infection, such as SARS-CoV-2. Therefore, in some embodiments, the subject or infant is monitored for progression to clinically significant coronavirus infection or disease based on an elevated level of afucosylated Fc glycans in the biological sample from the subject or the infant’s mother, respectively. In particular embodiments, the subject or infant is hospitalized.

In some embodiments, an afucosylated Fc glycan level of at least 5 percent (of, e.g., antigen-specific IgG) indicates that monitoring is in order. In some embodiments, this threshold is set a 10 percent.

In some embodiments, treatment of clinically significant coronavirus infection or disease can include administering an inhibitor of FcyRIIA or FcyRIIIA receptor signaling.

### Vaccination Against Coronavirus Infection

The antibody analysis methods described herein are also useful in the context of vaccination against coronaviruses. The presence of preexisting afucosylated Fc glycans in IgG antibodies indicates increased susceptibility to clinically significant coronavirus infection or disease. This means that in individuals having elevated afucosylated Fc glycans in their IgG antibodies, the risks associated with vaccination against a coronavirus can, in many cases, outweigh the benefits. Accordingly, measurement of elevated afucosylated Fc glycans in IgG antibodies provides a means of identifying individuals who should not be vaccinated.

A novel method of vaccinating against a coronavirus, thus, entails vaccinating a pre-selected patient population. In some embodiments, a subject pre-selected for a coronavirus vaccination is one that has been determined not to have an elevated level of afucosylated Fc glycans in IgG antibodies in a biological sample from the subject. In some embodiments, e.g., where the subject is an infant, if the mother of infant has been determined not to have an elevated level of afucosylated Fc glycans in IgG antibodies in a biological sample from the mother, the infant subject is pre-selected for a coronavirus vaccination. The antibody analysis in this context can be carried out essentially as described above.

In some embodiments, the measurement of elevated afucosylated Fc glycans among IgG that is specific for the coronavirus that is the target of the vaccine is the most predictive measurement.

### Inhibiting FcyRI, FcyRIIA or FcyRIIIA Receptor Signaling to Treat Infection and/or Reduce an Immune Response

Afucosylated Fc glycans increase affinity of IgGs for Fc receptors that activate immune responses. Because FcyRIIA or FcyRIIIA receptor are understood to be activating receptors generally, the work described herein indicates that inhibiting the binding of afucosylated Fc glycans to one or both of these receptors can be beneficial in other conditions in which afucosylated Fc glycans are present and an immune response plays a role in pathology. Examples of such conditions include autoimmune disorders, as described in Seeling, et. Al. (2017), Differential antibody glycosylation in autoimmunity: sweet biomarker or modulator of disease activity, Nature Reviews/Rheumatology, 13 :621-630 (which is hereby incorporated by reference for this description).

Accordingly, in some embodiments, a method of treating a subject acutely infected with a coronavirus and at risk of progression to clinically significant coronavirus infection or disease can include an inhibitor of FcyRIIA or FcyRIIIA receptor signaling to the subject. Suitable subjects for this treatment method include those described above, especially those identified as susceptible to progression to clinically significant coronavirus infection or disease, e.g., based on elevated Fc glycans, e.g., using any of the methods described herein. In some embodiments, the subject is one that has been tested and found to have a coronavirus infection. In some embodiments, this approach to treatment is combined with monitoring or one or more of the other approaches to treatment described herein.

In some embodiments, a method of reducing an immune response in a subject in need thereof can include administering an inhibitor of FcyRIIIA receptor signaling to the subject. Suitable subjects for this treatment method include those described above, e.g., those who have elevated afucosylated Fc glycans along with one or more symptoms of a pathological immune response. Examples of suitable subjects include those who have one or more autoimmune disorders, such as, e.g., Addison disease, Celiac disease, Dermatomyositis, Graves disease, Hashimoto thyroiditis, Multiple sclerosis, Myasthenia gravis, Pernicious anemia, Sjogren syndrome, Systemic lupus erythematosus, Type 1 diabetes, or one of the other at least 80 autoimmune disorders that have been described.

Because both FcyRIIA and FcyRIIIA receptor signaling can play a role in the pathology of the conditions described above, the inhibitor administered to the subject can inhibit FcyRIIA, but not FcyRIIIA, and vice versa. Alternatively, the inhibitor can be one that inhibits signaling through both receptors, or two different inhibitors, one for FcyRIIA and one for FcyRIIIA can be co-administered. Any means for inhibiting receptor signaling that the subject can tolerate well can be employed in these methods, including, e.g., gene knockout, a nucleic acid inhibitor, a protein inhibitor, or a small-molecule inhibitor.

### Inhibition of FcyRIIA and/or FcyRIIIA Receptor Signaling by Reducing the Level of Afucosylated Fc Glycans

Pooled human serum IgGs have been used in the treatment of chronic inflammatory and autoimmune diseases (intravenous immunoglobulin; IVIG therapy) since the 1980s, when it was discovered that high doses (1-3 g/kg body weight) serum IgG preparations could be used to treat immunothrombocytompenia in children. A similar approach could be taken in treating in the treatment methods described herein, except that the therapeutic IgG antibodies administered have a higher level of fucosylated Fc regions than the biological sample from the subject. Without being bound by any particular theory, it is believed that adding fucosylated IgG antibodies to the subject’s circulation effectively reduces the level of afucosylated IgGs, resulting in less signaling through FcyRIIA and/or FcyRIIIA. Because the Fc glycan plays a role in in this immunomodulation, fucosylated Fc regions could, in some embodiments, be administered, rather than full IgGs. In some embodiments, the level of afucosylated Fc glycans in a subject’s circulation can be lowered by obtaining IgG antibodies from the subject’s circulation, treating these IgG antibodies exogenously to fucosulate them, and returning the fucosylated IgG antibodies to the subject’s circulation.

Where the antigen(s) eliciting a pathological immune response are known, in some embodiments, the fucosylated IgG administered is specific for the antigen(s). When treating a subject infected with a coronavirus, anti-coronavirus IgG antibodies (i.e., specific for one or more coronavirus antigens) can be administered. In variations of such embodiments, one or more doses of fucosylated, neutralizing anti-coronavirus IgG can be administered. This latter approach is useful, for example, for treating an infant. In the infant, as the maternal neutralizing antibodies decay due to regular IgG half-life, this is the time of risk for enhanced infant disease. Administering a dose of neutralizing IgG of high enough titer to outlast any afucosylated maternal IgG can reduce the risk of this disease enhancement. In some embodiments, fucosylated IgGs (or Fc regions) can be administered to a mother who’s infant would be at high risk for disease during SARS-CoV-2 infection or during acute SARS-CoV-2 infection.

In general, when treating a subject with fucosylated IgG antibodies (or Fc regions thereof), the higher the level of fucosylated IgG antibodies in the preparation, the better. In various embodiments, the preparation has greater than 90, 91, 92, 93, 94, 95, 96, 97, 98, 99, or having 100 percent fucosylated antibodies. In various embodiments, IgG (or Fc region) dose is on the order of 0.1 mg/kg to 2 g/kg, 1.0 mg/kg to 1.75 g/kg, 1.0 mg/kg to 1.5 g/kg, 100 mg/kg to 1.25 g/kg, 500 mg/kg to 1.0 g/kg, or 750 mg/kg subject body weight.

Illustrative regimens for treating a subject with fucosylated IgG antibodies (or Fc regions thereof) include: 1, 2, or 3 times per day for a period of 1, 2, 3, 4, or 5 days.

Cell lines that produce IgG antibodies that are substantially all fucosylated in the Fc region are known and could be engineered to produce the desired IgG antibodies. For example, one or more broadly neutralizing anti- SARS-CoV-2 antibodies could be expressed in a cell line such as HEK 239T cells.

### Inhibition of Binding Between the Fc Region of IgG Antibodies and the FcyRIIA or FcyRIIIA Receptor

In some embodiments, the inhibitor inhibits binding between the Fc region of IgG antibodies and the FcyRIIA or FcyRIIIA receptor. Antibody inhibitors can be generated against the FcyRIIA or FcyRIIIA receptors, or antibodies that specifically bind afucosylated Fc regions can be produced. When the subject is human, the inhibitor of FcyRIIA or FcyRIIIA receptor signaling can be a humanized antibody. Methods for producing therapeutic antibodies are well known to those of skill in the art.

### Inhibition of Expression of the FcyRIIA or FcyRIIIA Receptor

Another approach to inhibiting signaling though the FcyRIIA and/or FcyRIIIA receptors is to down-regulate the expression of one or both of these receptors. Various standard techniques are available for reducing or blocking the expression of any gene whose sequence is known. These include, for example, genome editing techniques (such as CRISPR), as well as oligonucleotide-based techniques like siRNA and antisense methods. For known nucleotide sequences, one of skill in the art can readily use any of these techniques to down-regulate expression or knock out the gene. The nucleotide sequences for multiple alleles and splice variants of these receptors are known. For example, FcyRIIa has 2 major alleles that are referred to as H131 and R131, and FcyRIIIa has 2 major alleles that are referred to as V158 and F158. Sequence information is publicly available, and examples are given below.

### Inhibition of FcyRI Signaling

The above discussion of methods of inhibiting Fey receptor signaling also applies to inhibition of the FcyRI receptor, which also plays a role in coronavirus infection.

### Inhibition of ITAM-Mediated Signaling

All three of the Fey receptors discussed herein activate the immunoreceptor tyrosine-based activation motif (ITAM)-mediated signaling pathway, and this pathway is implicated in coronavirus infection generally and in progression to clinically significant coronavirus infection or disease. In particular, Example 4 demonstrates that two components of this pathway, spleen tyrosine kinase (Syk) and nuclear transcription factor of activated T cells (NFAT), play a role in the signaling of these Fey receptors during coronavirus infection. Other illustrative components of the pathway from Syk to NFAT that represent targets for coronavirus therapies include: Bruton’s tyrosine kinase (BTK), B-cell linker (BLNK), SRC-homology-2-domain-containing leukocyte protein of 76 kDa (SLP76), phospholipase Cy (PLCy), and calcineurin.

### Types of Inhibitors

The methods described herein can employ any type of inhibitor that can be administered to a subject, including, e.g., nucleic acids, polyclonal or monoclonal antibodies, antibody fragments that include the Fc region, antibody variants (e.g., humanized antibodies), as well as peptides, peptide analogs, and small molecules.

Peptide analogs are commonly used in the pharmaceutical industry as non-peptide drugs with properties analogous to those of the template peptide. These types of non-peptide compound are termed “peptide mimetics” or “peptidomimetics”. See Fauchere, 1986, Adv. Drug Res. 15 :29; Veber & Freidinger, 1985, TINS p.392; and Evans et al, 1987, J. Med. Chem. 30: 1229, which are incorporated herein by reference for their descriptions of peptide mimetics. Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide {i.e., a polypeptide that has a biochemical property or pharmacological activity), such as human antibody, but have one or more peptide linkages optionally replaced by a linkage selected from: —CH₂— H—, —CH₂—S—, —CH₂—CH₂—, —CH═CH—(cw and trans), —COCH₂—, —CH(OH)CH₂—, and —CH₂SO—, by methods well known in the art. Systematic substitution of one or more amino acids of a consensus sequence with a D-amino acid of the same type {e.g., D-lysine in place of L-lysine) may be used in certain embodiments to generate more stable peptides. In addition, constrained peptides comprising a consensus sequence or a substantially identical consensus sequence variation may be generated by methods known in the art (Rizo & Gierasch, 1992, Ann. Rev. Biochem. 61 :387, incorporated herein by reference for any purpose); for example, by adding internal cysteine residues capable of forming intramolecular disulfide bridges which cyclize the peptide.

Small-molecule inhibitors are available for Bruton’s tyrosine kinase (BTK), B-cell linker (BLNK), phospholipase Cj (PLOy), calcineurin, and NFAT, in addition to Syk (see Example 4). BTK inhibitors include, for example, ibrutinib (PCI-32765), acalabrutinib, ONO-4059, spebrutinib (AVL-292, CC-292), BGB-31 1 1, and HM71224. Phospholipase C inhibitors include aminosteroid (U73122), and edelfosine (ET-180CH3). Calcineurin inhibitors include, for example, ciclosporin, voclosporin, pimecrolimus and tacrolimus.

mRNA sequence for the Homo sapiens Fc fragment of IgG receptor Iia (FCGR2A), transcript variant 1 is provided as NCBI Reference Sequence : M_001136219.1 (SEQ ID NO : 1 in WO/2019/083904, incorporated herein).

DNA sequence for Homo sapiens Fc fragment of IgG receptor Ilia (FCGR3A), RefSeqGene (LRG 60) on chromosome 1 is provided as NCBI Reference Sequence: NG 009066.1 (SEQ ID NO:2 in WO/2019/083904, incorporated herein)

### Administration and Formulations

Active agents (e.g., IgG or other antibodies, Fc regions, small molecules) described herein can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4^{th} Ed. N.Y. Wiley-Interscience.

Methods of formulating such derivatives are known to those of skill in the art. For example, the disulfide salts of a number of delivery agents are described in PCT Publication WO 2000/059863, which is incorporated herein by reference. Similarly, acid salts of therapeutic antibodies, peptoids, or other mimetics, and can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, gly colic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain acid addition salts of the active agents herein include halide salts, such as may be prepared using hydrochloric or hydrobromic acids.

Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. In certain embodiments, basic salts include alkali metal salts, e.g., the sodium salt, and copper salts.

For the preparation of salt forms of basic drugs, the pKₐ of the counterion is preferably at least about 2 pH lower than the pKₐ of the drug. Similarly, for the preparation of salt forms of acidic drugs, the pKₐ of the counterion is preferably at least about 2 pH higher than the pKₐ of the drug. This permits the counterion to bring the solution’s pH to a level lower than the ρH^ to reach the salt plateau, at which the solubility of salt prevails over the solubility of free acid or base. The generalized rule of difference in pKₐ units of the ionizable group in the active pharmaceutical ingredient (API) and in the acid or base is meant to make the proton transfer energetically favorable. When the pKₐ of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In various embodiments, preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alkyl, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants or prepared from an anhydride or an acid chloride by reaction with ammonia or a lower alkyl amine.

Active agents can be administered locally (e.g., topically) or systemically, depending on the indication. In various embodiments, administration is topical, transdermal, oral, buccal, sublingual, nasal (or otherwise inhaled), rectal, parenteral (e.g., intravenous), etc. The compositions can be administered in a variety of unit dosage forms depending upon the method of administration. Suitable unit dosage forms, include, but are not limited to powders, tablets, pills, capsules, lozenges, pulmonary dosage forms (e.g., pulmonary dosage forms such as solutions for nebulizers, micronized powders for metered-dose inhalers, and the like), suppositories, patches, nasal sprays, injectables, implantable sustained-release formulations, lipid complexes, etc.

The active agents described herein can also be combined with a pharmaceutically acceptable carrier (excipient) to form a pharmaceutical composition. In certain embodiments, pharmaceutically acceptable carriers include those approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, auxiliary agent or vehicle with which an active agent of the present invention is administered.

Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, BHT (butylated hydroxytoluene), chelating agents, low molecular weight proteins, protection and uptake enhancers such as lipids, compositions that reduce the clearance or hydrolysis of the active agents, or excipients or other stabilizers and/or buffers.

The active agent(s) can be formulated with other physiologically acceptable compounds, particularly for use in the preparation of tablets, capsules, gel caps, and the like can include, but are not limited to, binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active agent(s) and the resulting composition is compressed. If desired, the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, hydroxypropylmethylcellulose acetate succinate, and Eudragit (Evonik, Germany; methaciylic-acrylic copolymers).

Other physiologically acceptable compounds that can be included with the active agent(s) include wetting agents, emulsifying agents, dispersing agents or preservatives that are particularly useful for preventing the growth or action of microorganisms. Various preservatives are well known and include, for example, phenol and sorbic acid. One skilled in the art appreciates that the choice of pharmaceutically acceptable carrier(s), including a physiologically acceptable compound depends, for example, on the route of administration of the active agent(s) and on the particular physio-chemical characteristics of the active agent(s).

In certain embodiments, the excipients are sterile and generally free of undesirable matter. These compositions can be sterilized by conventional, well-known sterilization techniques.

The dosage of active agent(s) can vary widely, and will be selected primarily based on activity of the active ingredient(s), body weight and the like in accordance with the particular mode of administration selected and the patient’s needs. In various embodiments dosages can be provided ranging from about 0.1 or 1 mg/kg/day to about 50 mg/kg/day and sometimes higher. Typical dosages range from about 3 mg/kg/day to about 3.5 mg/kg/day, for example, from about 3.5 mg/kg/day to about 7.2 mg/kg/day, from about 7.2 mg/kg/day to about 11.0 mg/kg/day, or from about 11.0 mg/kg/day to about 15.0 mg/kg/day. In certain embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic and/or prophylactic regimen in a particular subject or group of subjects.

In various embodiments, the active agent(s) is present in the formulation at a concentration ranging from about 1 nM, to about 1, 10, or 100 mM, more preferably from about 1 nM, about 10 nM, about 100 nM, about 1 µM, or about 10 µM to about 50 µM, about 100 µM, about 200 µM, about 300 µM, about 400 µM, or about 500 µM, preferably from about 1 µM, about 10 µM, about 25 µM, or about 50 µM to about 1 mM, about 10 mM, about 20 mM, or about 5 mM, most preferably from about 10 µM, about 20 µM, or about 50 µM to about 100 µM, about 150 µM, or about 200 µM.

In certain embodiments, the active agents of this invention are administered to the oral cavity. This is readily accomplished by the use of lozenges, aerosol sprays, mouthwash, coated swabs, and the like.

In certain embodiments the active agents of this invention are administered systemically (e.g., orally, or as an injectable) in accordance with standard methods well known to those of skill in the art. In other preferred embodiments, the agents, can also be delivered through the skin using conventional transdermal drug delivery systems, or transdermal drug delivery systems utilizing minimally invasive approaches (e.g., in combination with devices enabling microporation of upper layers of skin). Illustrative transdermal delivery systems include, but are not limited to transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active agent(s)” that is ultimately available for delivery to the surface of the skin. Thus, for example, the “reservoir” may include the active agent(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the patch and provides the device with much of its flexibility. The material selected for the backing layer is preferably substantially impermeable to the active agent(s) and any other materials that are present.

Formulations for topical delivery include, but are not limited to, ointments, gels, sprays, fluids, creams, reconstituted extracellular matrix complexes, synthetic skin, and the like. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Active agents can also be delivered topically using reconstituted extracellular matrix complexes, such as Matrigel® (U.S. Pat. No. 4,829,000, which is incorporated by reference herein for its disclosure of these materials) or synthetic skin-type materials, such as those disclosed in International Pub. Nos. WO2015198002 and WO2013164635 and U.S. Pat. No. 9,514,658 (each of which is incorporated by reference herein for its disclosure of these materials).

In certain embodiments, one or more active agents of the present invention can be provided as a “concentrate”, e.g., in a storage container (e.g., in a premeasured volume) ready for dilution, or in a soluble capsule ready for addition to a volume of water, alcohol, hydrogen peroxide, or other diluent.

While the invention is described with respect to use in humans, it is also suitable for animal, e.g., veterinary use. Thus certain preferred organisms include, but are not limited to humans, non-human primates, canines, equines, felines, porcines, ungulates, largomorphs, and the like.

### Embodiments

Embodiment 1 : A method of analyzing antibodies, wherein the method includes determining the level of afucosylated Fc glycans in IgG antibodies in a biological sample from a subject acutely infected with SARS-CoV-2 or from a maternal subject having an infant acutely infected with SARS-CoV-2, wherein the subject has not been determined to have an autoimmune disorder.

Embodiment la: A method of determining the susceptibility of a subject to clinically significant COVID-19 infection or disease, the method comprising determining the level of afucosylated Fc glycans in IgG antibodies in a biological sample from the subject, wherein an elevated level of afucosylated Fc glycans in the biological sample indicates that the subject is susceptible to clinically significant COVID-19 infection or disease.

Embodiment 2: The method of embodiment 1 or la, wherein the IgG antibodies are IgGl antibodies.

Embodiment 3 : The method of embodiment 1 or la or embodiment 2, wherein the IgG antibodies are IgG antibodies of all specificities.

Embodiment 4: The method of embodiment 1 or la or embodiment 2, wherein the IgG antibodies are IgG antibodies specific for a SARS-CoV-2 antigen.

Embodiment 5: The method of embodiment 4, wherein the SARS-CoV-2 antigen is a spike protein antigen.

Embodiment 6: The method of embodiment 4, wherein the SARS-CoV-2 antigen is a full-length spike protein antigen.

Embodiment 7: The method of any one of embodiments 1-6, wherein the subject is a human.

Embodiment 8: The method of embodiment 7, wherein the subject has the acute SARS-CoV-2 infection in the presence of preexisting IgG antibodies that are reactive with the infecting SARS-CoV-2.

Embodiment 9: The method of embodiment 7, wherein the subject is a maternal subject having an infant.

Embodiment 9a: The method of embodiment 9, wherein the infant is acutely infected with a SARS-CoV-2.

Embodiment 10: The method of embodiment 9 or 9a, wherein the maternal subject has IgG antibodies that are reactive with the infecting SARS-CoV-2 .

Embodiment 11 : The method of any one of embodiments 1-10, wherein the biological sample is blood or a blood fraction.

Embodiment 12: The method of any one of embodiments 1-11, the method including monitoring the subject or infant for progression to clinically significant COVID-19 infection or disease based on an elevated level of afucosylated Fc glycans in the biological sample.

Embodiment 13 : The method of any one of embodiments 1-12, wherein the subject or infant is one that has tested positive for a SARS-CoV-2 infection.

Embodiment 14: The method of embodiment 12 or embodiment 13, wherein the method includes hospitalizing the subject or infant based on having an elevated level of afucosylated Fc glycans in the biological sample.

Embodiment 15: The method of any one of embodiments 12-14, wherein the clinically significant flaviviral infection or disease includes severe COVID-19 disease.

Embodiment 16: The method of embodiment 15, wherein the method includes treating the subject or infant to prevent or inhibit progression to, or to manage, severe COVID-19 disease, based on an elevated level of afucosylated Fc glycans in the biological sample.

Embodiment 17: The method of embodiment 16, wherein the treatment includes transfusion with blood and/or platelets.

Embodiment 18: The method of any one of embodiments 12-17, wherein the method includes administering one or more doses of neutralizing anti- SARS-CoV-2 IgG, wherein the percentage of fucosylated Fc regions in each dose is greater than 95%.

### EXAMPLES

Numerous embodiments of the present disclosure is demonstrated by the following non-limiting Example. Severe acute respiratory syndrome coronavirus 2 (SARS-CoV-2) infections can cause Coronavirus Disease 2019 (COVID-19), which manifests with a range of severities from mild illness to life threatening pneumonia and multi-organ failure. Severe COVID-19 is characterized by an inflammatory signature including high levels of the cytokines IL-6 and TNFa. Pathways that contribute to this inflammatory state are important to clarify in order to develop targeted therapies for COVID-19. As shown in the Example, severe COVID-19 patients produce a unique antibody signature, including significantly increased levels of IgG1 with afucosylated Fc glycans. This Fc modification on SARS-CoV-2 IgGs enhanced interactions with the activating FcyR, FcyRIIIa; when incorporated into immune complexes, Fc afucosylation enhanced production of inflammatory cytokines by monocytes, including IL-6 and TNFa. These results show that severe COVID-19 is associated with production of afucosylated Fc structures, an activating antibody modification that can promote production of cytokines associated with severe COVID-19.

### Example 1

### A. Severe COVID-19 Patients Display Specific IgG Fc Domains

To address whether there are specific features of antibodies produced by mild or severe COVID-19 patients, a multi-dimensional analysis of antibodies from severe (hospitalized) and mild (outpatient) SARS-CoV-2 infections was performed with a focus on antibody features that are known to augment effector functions. 43 hospitalized PCR+ COVID-19 patients, divided into those treated in the ICU (n=21) or on the floor (n=22), along with 18 PCR+ COVID-19 outpatients were studied. In addition, because children are rarely diagnosed with COVID-19, and almost never develop severe COVID-19 despite being susceptible to productive infections (Liu, W. et al., N Engl J Med 382, 1370-1371 (2020); Xu, Y. et al., Nat Med 26, 502-505 (2020); Bi, Q. et al., Lancet Infect Dis (2020); and Dong, Y. et al., Pediatrics (2020)) it was reasoned that it would be informative to study SARS-CoV-2 antibodies produced by children. To this end, approximately 800 remainder sera from pediatric patients in a large Northern Californian health care system were screened and identified 16 that were positive for antibodies against the receptor binding domain (RBD) of the SARS-CoV-2 spike protein; all positive samples from children were validated in a secondary screen against the full-length spike protein, as previously described (Stadlbauer, D. et al., Curr Protoc Microbiol 57, e100 (2020)).

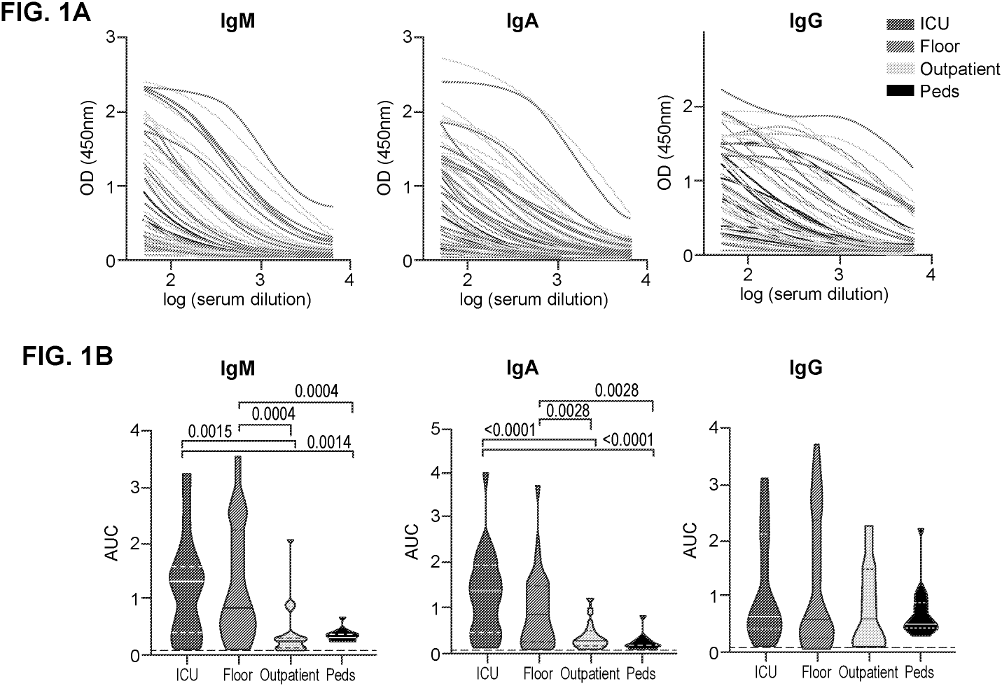

To characterize serologic correlates of disease severity, anti-RBD immunoglobulin isotype titers was first profiled in sera from COVID-19 patients or from seropositive children. Among the study subjects, severe COVID-19 patients (ICU and floor) had significantly higher serum titers of IgM and IgA RBD-binding antibodies compared to both mild COVID patients and seropositive children. The titers of anti-RBD IgG antibodies were not significantly different amongst the groups (FIGS. 1A, 1B).

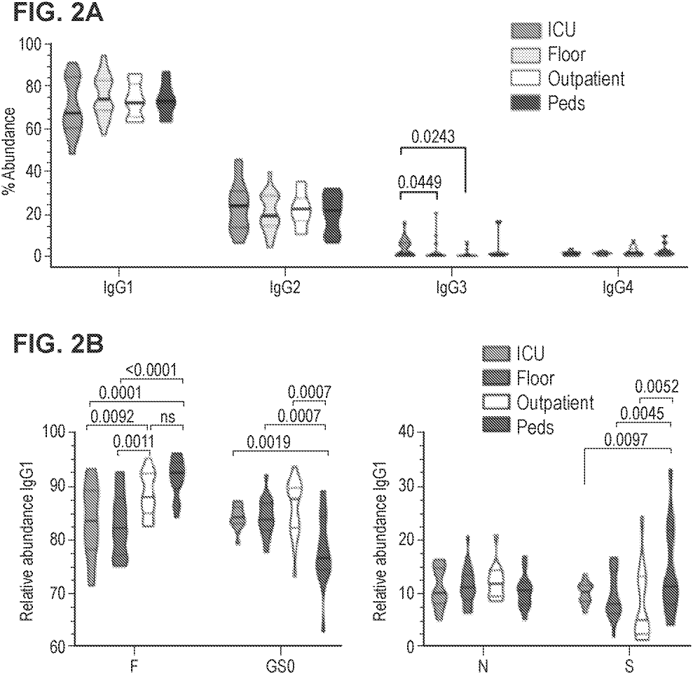

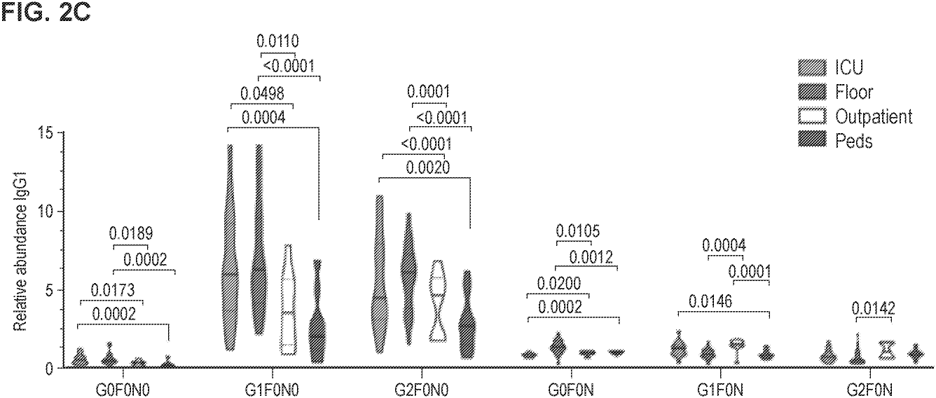

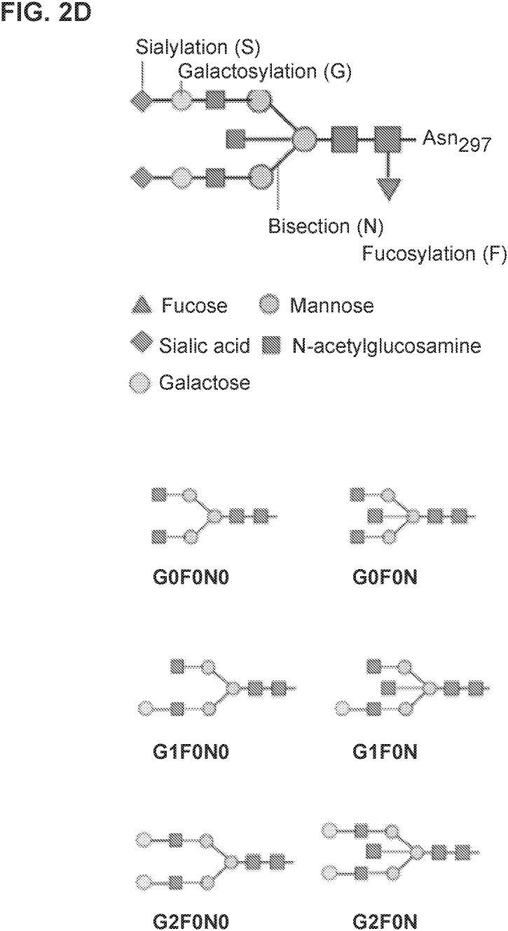

Next, the determinants of anti-RBD IgG effector function were defined in the COVID-19 patients and seropositive children. The absolute abundance of IgG subclasses was characterized by mass spectrometry. The abundance of various subclasses was similar between groups, with a small but significant increase in IgG3 produced by COVID-19 patients who were in the ICU (FIG. 2A). Anti-RBD IgG1, the most abundant IgG subclass, was next characterized for post-translational modifications of the Fc using well-established mass spectrometric methods (Thulin, N.K., Wang T.T., Cell Reports 31 (2020); Wang, T.T. et al., Cell 162, 160-169 (2015); and Wang, T.T. et al., Science 355, 395-398 (2017)). Notably, anti-RBD IgG1 from severe COVID-19 patients was significantly reduced in core fucosylation when compared with anti-RBD IgG1 from mild COVID-19 patients or from children (FIG. 2B). Of the six Fc glycoforms quantified which lack core fucose (afucosylated glycans), those without a bisecting N-acetyl glucosamine (FONO) were significantly increased in severe COVID-19 patients (FIG. 2C, D).

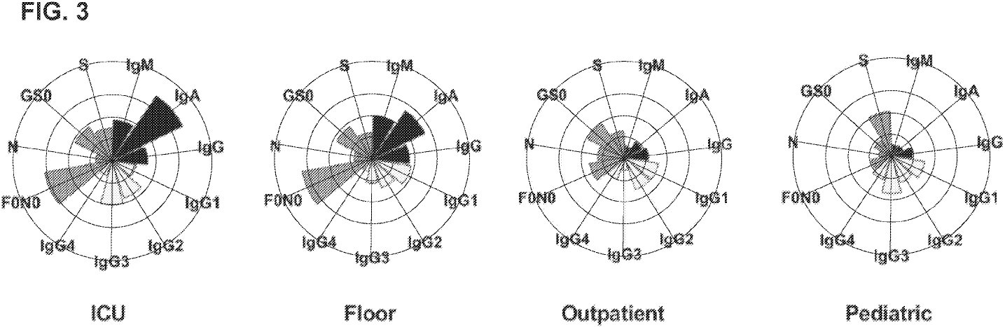

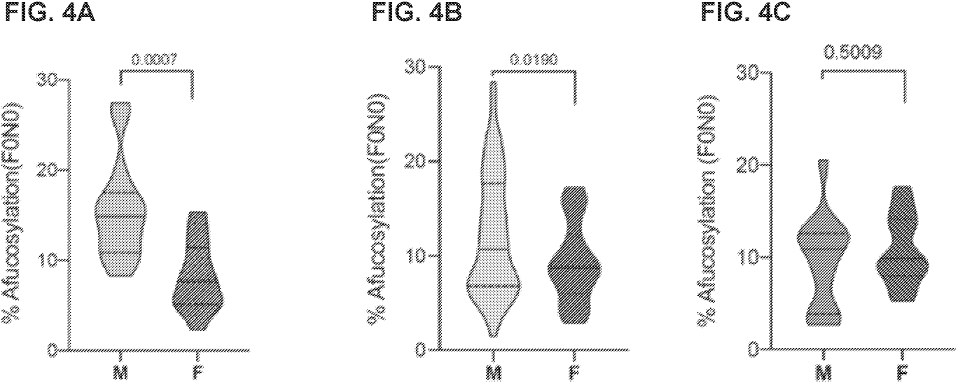

Overall, these data show a unique antibody signature associated with COVID-19 severity based on multiparametric characterization of SARS-CoV2-specific humoral responses comprising isotype titers, IgG subclasses and IgG1 Fc-glycan structures. Severe COVID-19 patients produced significantly higher titers of anti-RBD IgM and IgA isotype antibodies, increased IgG3 subclass (ICU patients) and increased afucosylated (FONO) Fc glycoforms relative to patients with mild COVID-19 (FIG. 3). The IgG3 subclass and F0N0 IgG1 modifications are features that increase Fc interactions with activating/inflammatory FcyRs (Wang, T.T., Curr Top Microbiol Immunol (2019)).

### B. Fc Afucosylation in Severe COVID-19 is Elevated in Males

To begin to dissect the cause of elevated levels of IgG1 afucosylation in severe COVID-19 patients, the question of whether infection itself triggered an observable change in Fc glycoforms was invesitigated. To do this, a longitudinal analysis of levels of F0N0 and other Fc glycoforms on RBD-reactive IgG1 was performed from paired sera drawn at acute (T1) and later time points. Paired samples were selected from individuals who were positive for IgG at T1 and late time points were drawn 2, 3- or 4-weeks post T1. No significant changes were observed in the levels of FONO, bisection or galactosylation over time but the amount of sialylation was significantly reduced between T1 and week-4 samples. This suggested that infection triggered an acute increase in Fc sialylation but not in other Fc glycoforms. In contrast to SARS-CoV-2 infection, we have previously observed that acute dengue virus infections can trigger production of highly afucosylated IgGs which declines in the weeks following infection (Wang, T.T. et al., Science 355, 395-398 (2017)). To further probe whether anti-RBD Fc glycoforms were associated with SARS-CoV-2 viral load, correlation analyses were performed between anti-RBD Fc glycoforms and SARS-CoV-2 RNA levels from nasopharyngeal swabs taken during acute infection. No correlation was observed between any Fc glycoforms and viral RNA load as determined by the cycle threshold (CT) value. In all, these data do not support that SARS-CoV-2 virus infection regulated Fc glycosylation during infection.

Studies have previously shown that antibody glycans may be influenced by age and sex (Bakovic, M.P. et al., J Proteome Res 12, 821-831 (2013); Chen, G. et al., J Proteomics 75, 2824-2834 (2012); de Haan, N., et al., J Proteome Res 15, 1853-1861 (2016); and Kapur, R. et al., Blood 123, 471-480 (2014)). A multivariate regression analysis was performed on samples from hospitalized COVID-19 patients from the Stanford Hospital Center (n=30, F=14, M=16) to determine whether age and/or sex may have played a role in regulating the abundance of Fc glycans. Interestingly, sex was significantly correlated with FONO glycosylation (p=0.0007) and age was not a confounding variable. Males had significantly higher levels of anti-RBD FONO Fc glycoforms over females (FIG. 4A). To determine the generalizability of this finding, we studied samples from a second cohort of hospitalized COVID-19 patients treated in Northern California Kaiser Permanente hospitals (n=81, M=55, F=26). In this larger cohort, males also had significantly elevated anti-RBD FONO Fc glycoforms over females (p=0.019) (FIG. 4B). A similar sex associated difference in levels of afucosylation was not observed in mild COVID-19 patients (n=27, F=14, M=13) (FIG. 4C). No other Fc glycoforms segregated by sex in any of the cohorts. Further, age did not correlate with any of the characterized anti-RBD Fc glycoforms. Overall, these data showed that males who were hospitalized with COVID-19 produced higher levels of afucosylated, anti-RBD IgG1 antibodies over females.

### C. Afucosylated SARS-CoV-2 Immune Complexes Can Promote FcyRIIIa Interactions and Inflammatory Cytokine Production

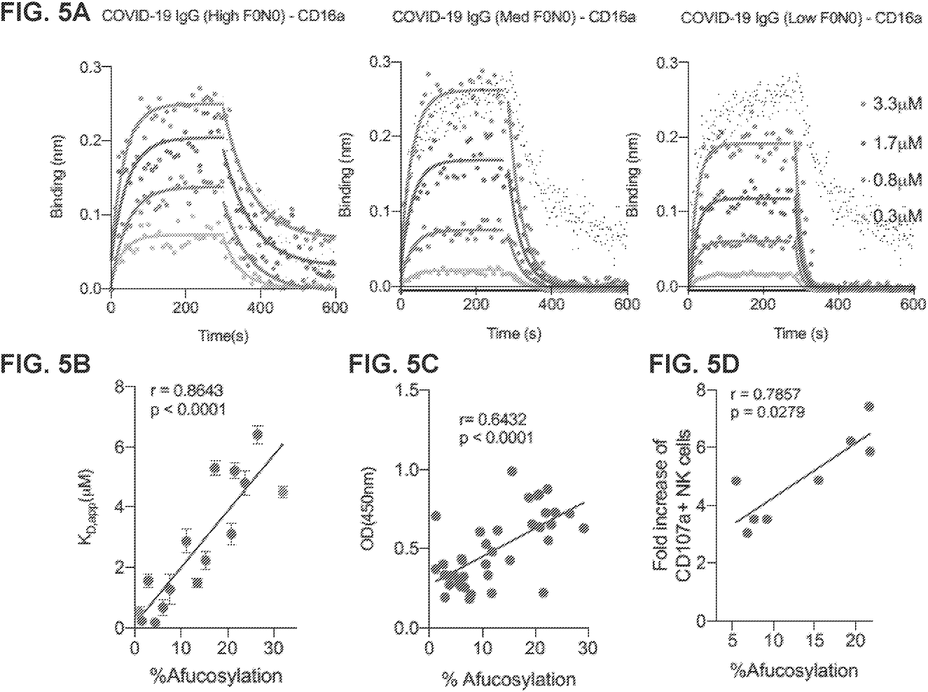

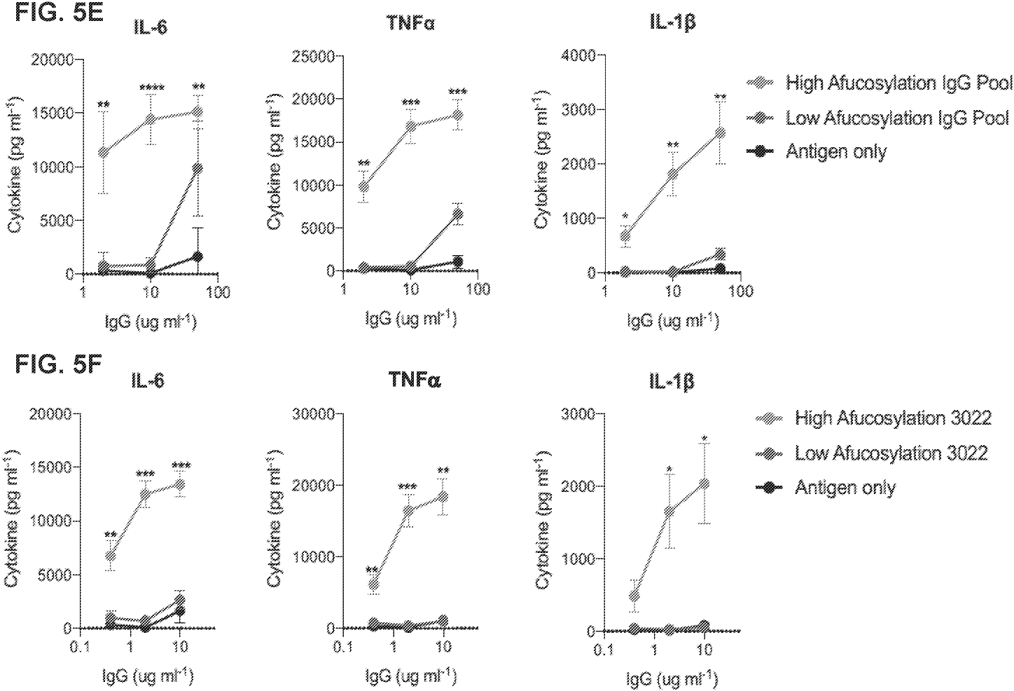

Afucosylation of IgG1 Fc confers 5-10-fold higher affinity for the activating FcyR, FcyRIIIa on a monomeric basis (and higher affinity in the context of a multivalent complex), relative to fucosylated IgG1 (Li, T. et al., Proceedings of the National Academy of Sciences of the United States of America 114, 3485-3490 (2017)). FcyRIIIa is present on monocytes, macrophages and NK cells at baseline and can enhance cell activation, pro-inflammatory cytokine production and cytotoxic effector cell activity (Kramer, P.R., et al., Eur J Immunol 39, 561-570 (2009); and Bournazos, S., et al., Microbiol Spectr 4 (2016)). Thus, distinct levels of fucosylation within SARS-CoV-2 immune complexes would be expected to modulate their binding to FcyRIIIa and activating ITAM signaling (Li, T. et al. Proceedings of the National Academy of Sciences of the United States of America 114, 3485-3490 (2017)). To determine whether serum IgGs from study subjects differed in their capacity to bind FcyRIIIa, the apparent dissociation constant of purified serum IgGs from severe COVID-19 patients with a range of fucose levels was measured, to recombinant FcyRIIIa (F158). IgGs from patients with RBD IgG1 afucosylation >20% conferred ~3-fold higher affinity to FcyRIIIa over IgGs with medium afucosylation and 5-6 fold over IgGs with low IgG1 afucosylation (<10%). Further, RBD IgG1 afucosylation levels correlated with the apparent dissociation constant (Kd) of IgGs for FcyRIIIa (p<0.0001)(FIGS. 5A, B). To further characterize this using homogenous Fc structures, the anti-SARS-CoV2 RBD monoclonal antibody (mAb) CR3022 (Yuan, M. et al., Science 368, 630-633 (2020)) was expressed as a fully fucosylated IgG1 or as a variant with 32.6% afucosylated Fc, as determined by mass spectrometry; these variants differed in apparent kD for FcyRIIIa by ~7-fold (FIG. 5B). Next, an ELISA was performed to measure binding by IgGs from individual sera to FcyRIIIa. 38 sera were selected that represented the range of afucosylation over the sample set. Consistent with the binding results using purified IgGs, the amount of anti-RBD IgG1 afucosylation correlated with binding to FcyRIIIa (p<0.0001) (FIG. 5C).

To determine whether these binding differences were physiologically relevant to activation of primary immune cells, in vitro stimulation assays were performed. First, healthy donor NK cell responses to immune complexes composed of patient IgGs and RBD were assessed. NK cells enable convenient evaluation of FcyRIIIa-dependent antibody signaling and effector function because they express only the FcyRIIIa and no other FcyRs. NK cell degranulation as measured by CD107a-positive staining correlated with the abundance of anti-RBD afucosylation in immune complexes (p=0.0279) (FIG. 5D). Given the prominent role of proinflammatory cytokines in severe COVID-19 (Chen, G. et al., The Journal of clinical investigation 130, 2620-2629 (2020); Del Valle, D.M. et al., Nature medicine (2020); and Qin, C. et al., Clinical infectious diseases : an official publication of the Infectious Diseases Society of America (2020)), the potential of immune complexes with different levels of Fc afucosylation was also assessed to stimulate cytokine production by primary monocytes. Immune complexes were formed from pooled IgG mixed with SARS-CoV-2 pseudoparticles; these complexes were added to primary cells from separate donors. IgG pools were derived from patients with high (>20%) or low (<10%) anti-RBD IgG1 afucosylation. Highly afucosylated immune complexes elicited increased production of proinflammatory cytokines, principally IL-6, TNF-α and IL-1β compared to immune complexes with low afucosylation (FIG. 5E). To better define the role of Fc fucosylation alone on the differential activation of monocytes, this assay was performed with immune complexes made from SARS-CoV-2 pseudoparticles along with mAb 3022 variants that differed only in Fc fucosylation, as described above. Cytokines including IL-6, TNF-α and IL-1β were significantly enhanced after incubation of monocytes with afucosylated mAb 3022 complexes (FIG. 5F).

### D. Antibody Quality and Dynamics and Predictability of Disease Progression

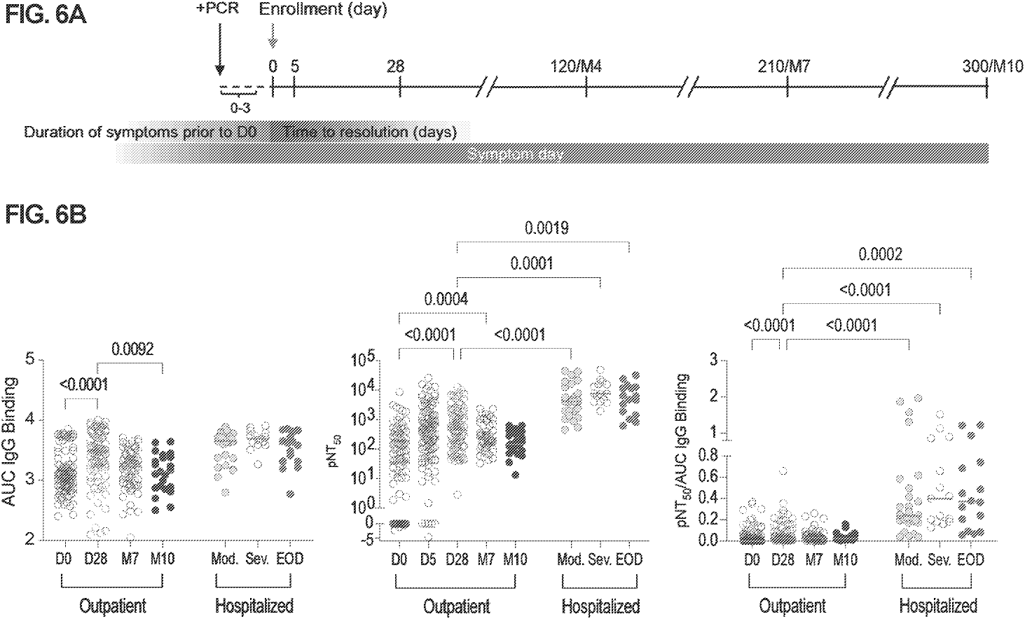

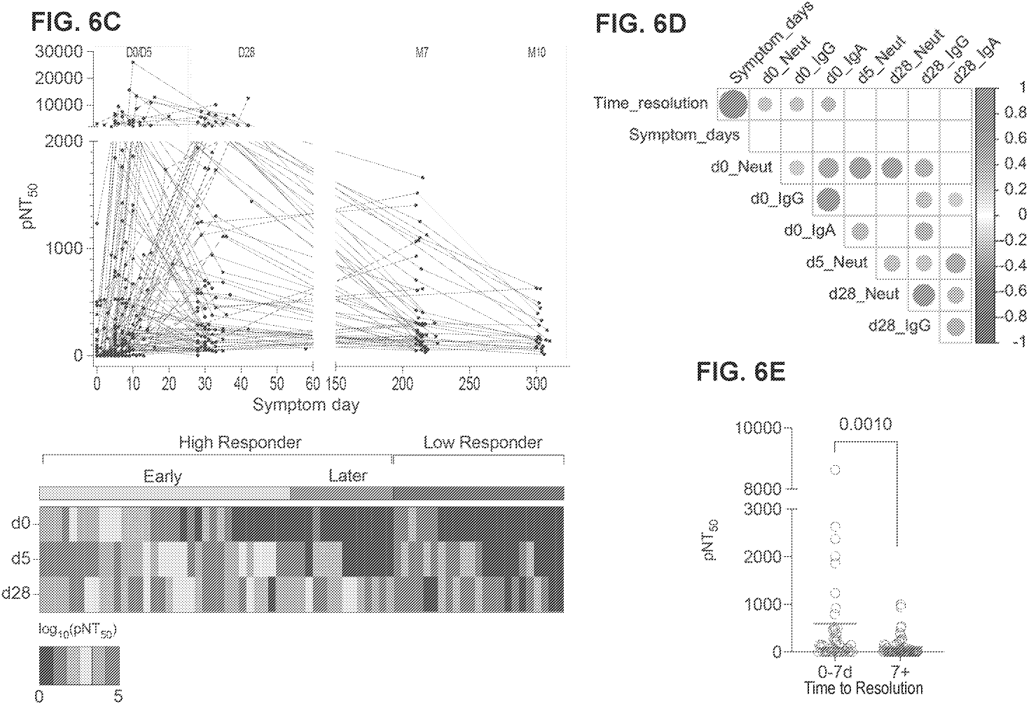

FIG. 6 provides antibody quality and dynamics in a longitudinal cohort of COVID-19 outpatients. Serological analyses were performed on longitudinal samples from a cohort of COVID-19 outpatients (n=120) collected at day 0 (D0, enrollment), day 5 (D5), day 28 (D28), month 7 (M7) and month 10 (M10). All the participants were positive by PCR before enrollment and the mean duration of symptoms prior to enrolment was 5 days (Duration of symptoms prior to Day 0) (FIG. 6A). SARS-CoV-2 full length spike (S) binding IgG (AUC), half-maximal SARS-CoV-2 pseudovirus neutralizing titers (pNT50) and the ratio of normalized neutralizing to IgG binding titers are shown in FIG. 6B. For the outpatient cohort, levels of both binding and neutralizing antibodies showed significant increase over time from day 0 to the day 28 timepoint (p<0.0001). The antibody response was robust till 7 months (n=65) post enrollment and showed a decrease in binding titers (p=0.0092) and a modest but non-significant decrease in neutralization only at the M10 (n=23) time point. As compared to COVID-19 outpatients, hospitalized patients across a spectrum of disease severity ranging from moderate (Mod. n=28) to severe with (EOD, n=16) or without (Sev. n= 15) EOD had significantly elevated levels of both neutralizing (p=<0.0001 D28 vs Mod., p=0.0001 D28 vs Sev, p=0.0019 D28 vs EOD) and neutralizing to binding (IgG) antibodies (p=<0.0001 D28 vs Mod., p=<0.0001 D28 vs Sev, p=0.0002 D28 vs EOD).

The kinetics of neutralizing antibody response over time has been plotted for the outpatient cohort. pNT50 followed two basic patterns over time; in one group (termed as low responder), the pNT50 was below 500 for the duration of the study, while in the other group (termed high responder), pNT50 was greater than 500 but peak neutralizing titers were achieved at two distinct time periods: early after enrollment (D0/D5) (early), or by study day 28 (later) (FIG. 6C). In FIG. 6D, the cross-correlation matrix shows the relationship between multiple features of the antibody response (D0, D5 and D28 pNT50, D0 and D28 IgG and IgA titers) in longitudinally analyzed COVID-19 patient samples. Only early neutralization and binding titers correlated with a shorter course of disease. Data is shown only for statistically significant (p < 0.05) correlations. As shown in FIG. 6E, early high responders in the outpatient cohorts who elicited neutralizing titers within the first 15 days of symptoms had a significantly shorter course of disease (p=0.001).

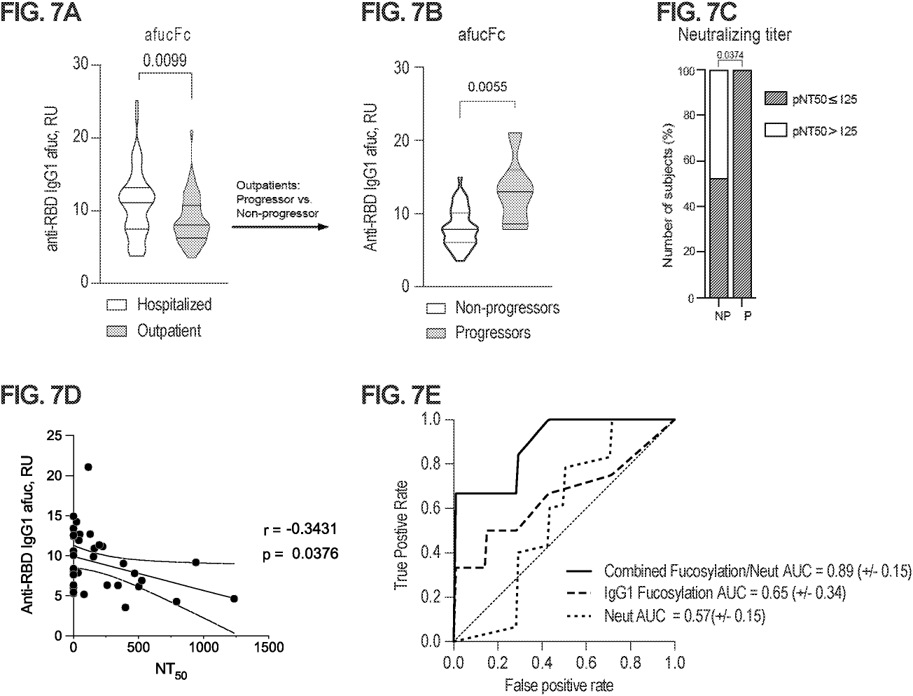

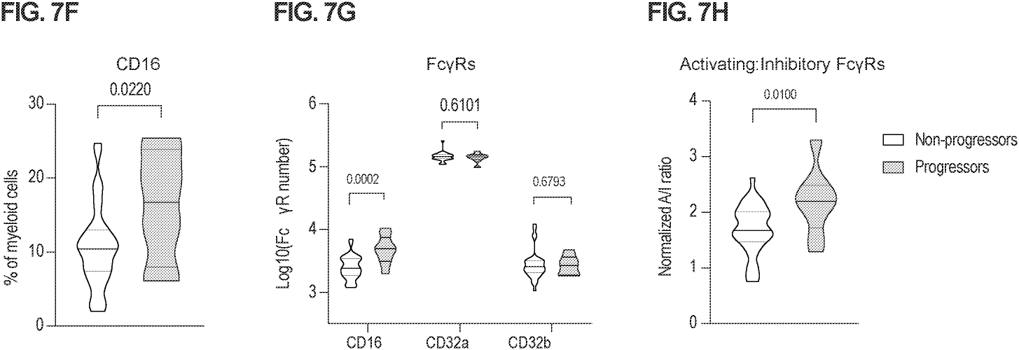

As shown in FIG. 7, low early neutralizing titers and elevated Fc afucosylation predict disease progression. Anti-RBD IgG1 Fc afucosylation (afuc) was characterized (n=?). As shown in FIG. 7A, patients who were hospitalized had significantly higher Fc afucosylation when compared with anti-RBD IgGls from the outpatient cohort (p=0.0099). Subjects (n=8) within the cohort whose symptoms progressed over time post enrollment requiring an emergency room visit or hospitalization (Progressors) had significantly higher afucosylation than subjects who maintained their asymptomatic or mild status (Non-progressors) (p=0055) (FIG. 7B). FIG. 7C shows the distribution of early neutralizing titers (D0/D5) amongst Progressors (P) and Non-progressors (NP) showed a statistically significant difference (p=0.0374, Fisher’s exact test). FIG. 7D shows the correlation between anti-RBD IgG1 afucosylation and D0 neutralization titers have been plotted. IgG1 afucosylation is inversely correlated with D0 pNT50 (Pearson’s correlation coefficient r=-0.3431, p=0.0376).

As shown in FIG. 7E, mean ROC response and the area under the curve (AUC) with its standard deviation obtained using random forest classifier with 6-fold cross validation has been plotted. Individually, afucosylation had modest (AUC=0.65 +/- 0.34) and early neutralization titers minimal (AUC=0.57 +/- 0.15) predictive powers, while combining the two features could robustly separate progressors from non-progressors with high predictive accuracy (AUC=0.89 +/- 0.15). PBMCs from subjects isolated on D0 were assessed by flow cytometry for CD16+ monocyte frequencies as a percent of total CD11c+ HLA-DR+ lin- myeloid cells. Progressors had significantly higher CD16+ monocytes than the non-progressors (p=0.0220) as shown in FIG. 7F. In FIG. 7G, quantitative flow cytometry was employed to determine Feγ receptor (FcyR) expression on bulk myeloid cells. Progressors had increased CD16 expression in the myeloid compartment as compared to non-progressors (p=0.0002). Other FcyR expression (CD32a and CD32b) levels were not significantly different between the two groups. Finally, as shown in FIG. 7H, the activating to inhibitory ratio (A/I) which was calculated by combining the range normalized, quantitative expression of FcyRs CD16, CD32a and CD32b has been plotted. Progressors had significantly higher A/I as compared to non-progressors (p=0.01).

### E. Discussion

Overall, these aforementioned studies and present disclosure show that a specific IgG1 Fc structure, characterized F0N0 glycoform modification, is elevated in severe COVID-19. This was in contrast to patients with mild symptoms and seropositive children. Further, differences in core IgG1 afucosylation impacted FcyRIIIa binding and, within SARS-CoV-2 immune complexes, enrichment of afucosylated Fc structures could promote production of cytokines including IL-6, TNF-α and IL-1β by primary monocytes.

The present disclosure addresses the antibodies that are naturally produced in SARS-CoV-2 infections. The finding that afucosylated IgG1 Fc structures are significantly enriched in severe disease suggests this modification may be a biomarker for risk.

The present disclosure also reports that male sex was associated with high Fc afucosylation in severe COVID-19. It is well established that men are disproportionately affected by COVID-19, including constituting approximately two-thirds of COVID-19 patients and generally having worse clinical outcomes (Guan, W.J. et al., N Engl J Med 382, 1708-1720 (2020); Richardson, S. et al., JAMA (2020); Sharma, G., Volgman, A.S. & Michos, E.D., JACC Case Rep (2020); and Chakraborty, S. et al. medRxiv (2020)). Though multiple factors are certain to contribute to the correlation between poor outcomes in COVID-19 and male sex (Takahashi, T. et al., Nature (2020)), the present disclosure suggests that overproduction of afucosylated Fc structures in males and an associated activation of inflammatory FcyR pathways is one “hit” that can promote progression to severe COVID-19.

### E. Methods

### Cloning, Expression and Protein Purification

The His₆-tagged SARS-CoV-2 RBD construct was a kind gift from Florian Krammer. The full length recombinant SARS-CoV-2 spike protein (residues 1-1208 (GenBank:MN908947)) construct was designed with the following modifications: two well-characterized proline substitutions ( K986P and V987P) (Pallesen, J. et al., Proc Natl Acad Sci U S A 114, E7348-E7357 (2017)); a four amino acid substitution to remove the furin cleavage site (RRAR → GSAS) in order to stabilize the pre-fusion conformation (Wrapp, D. et al., Science 367, 1260-1263 (2020)); a synthetic trimerization motif- the globular β-rich ‘foldon’ from T4 fibritin to promote oligomerization in lieu of the native trans-membrane (TM) domain; a human rhino virus 3C (HRV 3C) protease cleavage site; a C-terminal Hiss tag. Mammalian codon-optimized gene fragments were synthesized (Integrated DNA Technologies, Inc.) and cloned using Gibson Assembly (New England BioLabs) into a CMV/R promoter driven mammalian expression vector between XbaI and BamHI restriction sites.

Both the constructs were transiently transfected into Expi293F cells (Thermo Fisher Scientific) as per the manufacturer’s recommendation. Briefly, Expi293F cells at a density of 3×10⁶ viable cells/ml maintained in Expi293 expression medium (Thermo Fisher Scientific) were transfected with expression plasmids complexed with ExpiFectamine 293 transfection reagent. 18 hours post-transfection, the cells were supplemented with a cocktail of transfection enhancers. The cultures were incubated for four days, following which the culture supernatants were harvested by centrifugation for protein purification. The supernatants were incubated with phosphate-buffered saline (PBS (Gibco))-equilibrated Ni-nitriloacedic acid (NTA) resin (GE HealthCare) for 2 h at 4° C. under mild-mixing conditions to facilitate binding. The proteins were subsequently eluted using 500 mM imidazole (in PBS, pH 7.4) under gravity flow. The eluted fractions were pooled, buffer exchanged into PBS (pH 7.4) and concentrated using Amicon Ultra centrifugal units (EMD Millipore) to a final concentration of ~1 mg/ml. Protein purity was assessed by sodium dodecyl sulfate-polyacrylamide gel electrophoresis (SDS-PAGE). Size exclusion chromatography was used to determine the oligomeric state of the purified proteins under non-denaturing conditions at room temperature on a Superdex-200 analytical gel filtration column (GE HealthCare). For molecular weight estimations, the column was calibrated using broad range molecular weight markers (GE HealthCare).

MAb 3022 (high and low afucosylation) were produced from Expi293F cells (Thermo Fisher Scientific) as described above. For the afucosylated 3022 production, 200 µM of fucosyl transferase inhibitor 2F-Peracetyl-Fucose (Sigma Aldrich) was added to the culture after transfection. Culture supernatants were harvested 5 days after transfection and antibody purifications were done over Protein G Sepharose 4 Fast Flow resin (GE Healthcare). The antibodies were buffer exchanged into PBS pH 7.4 and concentrated using Amicon Ultra centrifugal units (EMD Millipore). Afucosylation levels of the mAbs were analysed by mass spectrometry as described herein.

### ELISAs

### Screening ELISA

A rapid, high-throughput screening ELISA was performed on a total of 789 pediatric samples to test seropositivity following a modified version of a protocol described previously. Briefly, round bottom 96 well plates (Immunolon 2HB (Thermo Scientific)) plates were coated with 50 µl of RBD at 2 µg/ml in PBS for 1h at room temperature (RT). Next, the plates were blocked for an hour with 3% non-fat milk in PBS with 0.1% Tween 20 (PBST). All serum samples from COVID-19 patients, the pediatric cohort and the negative controls were heated at 56° C. for 1 h, aliquoted and stored at -80° C. For the first round of screening, all samples were diluted 1:50 in 1% non-fat milk in PBST. 50 µl of the diluted sera was added to each well and incubated for 2 h at RT. Following primary incubation with the sera, 50 µl of 1:5000 diluted horse radish peroxidase (HRP) conjugated goat anti-Human Ig Fab (Southern Biotech) was added and incubated for 1 h at RT. The plates were developed by adding 50ul/well of the chromogenic substrate 3,3′,5,5′-tetramethylbenzidine (TMB) solution (Millipore Sigma). The reaction was stopped with 0.2 N sulphuric acid (Sigma) and absorbance was measured at 450 nm (SPECTRAmax 250, Molecular Devices). The plates were washed 5 times with PBST between each step and an additional wash with PBS was done before developing the plates. Samples were considered seropositive against RBD if their absorbance value was greater than the mean plus four standard deviation (SD) of all negative controls (n=130).

### Validation ELISA of Seropositive Pediatric Samples

The serum samples from the pediatric cohort, which showed seropositivity against RBD were validated by a second round of screening against the full-length SARS-Cov-2 spike protein (S). As described above, plates were coated with 50 ul of 2 ug/ml S protein in PBS. Following blocking and wash, the plates were incubated with a 5-fold dilution series of RBD positive sera (50µl) starting at 1:50 for 2 h at room temperature. All the subsequent steps were followed as described above. Sera from children and COVID-19 patients were considered positive if they reached a threshold of the average value of 130 historical negative controls plus six standard deviations. The specificity of the assay was also tested on control sera from 12 subjects with documented seasonal coronavirus infections collected in early 2019.

### Isotyping by ELISA

Sera were diluted 5-fold starting at 1:50 and ELISAs were performed as described above. The various secondary antibodies used for isotyping were 1:5000 dilutions of HRP-conjugated Goat Anti-Human IgG Fc (Southern Biotech), Mouse Anti-Human IgM (Southern Biotech) and Goat Anti-Human IgA Fc (Southern Biotech).

### CD16a ELISA

Human recombinant CD16a (Sino Biological) was immobilized at 3 ug/ml (50 ul/well) in PBS at 4° C. overnight, followed by an hour of blocking with 3% non-fat milk in PBST. 50 ul of 1:50 diluted sera from pediatric seropositive (n=11), PCR+ and seropositive (n=22) and healthy controls (n=5) were added to each well and incubated for 2 h at 37° C. Subsequently the plates were incubated for 1 h at 37° C. with 1:5000 dilution of HRP-conjugated Goat Anti-Human IgG Fc (Southern Biotech) secondary antibody, developed as described previously with TMB and absorbance was recorded at 450 nm.

### Clinical Cohorts and Samples

Remnant sera from pediatric subjects and from PCR+ COVID-19 patients were obtained from Kaiser Permanente Northern California. The sera were collected for a variety of clinical tests at one of 75 distinct hospitals or outpatient clinics across 17 counties in Northern California between Mar. 30, 2020 to Apr. 19, 2020. Additional serum samples from PCR+ COVID-19 patients were from the Stanford ICU Biobank (protocol #28205) or from (protocol #NCT04331899) (Wilk AJ*, et al., Nature Medicine, IN PRESS (2020)). Characterization of these samples was performed under a protocol approved by the Institutional Review Board of Stanford University (protocol #55718).

Samples from people with seasonal coronavirus infections were collected at the University of Chicago. Samples were de-identified serums of healthcare workers that had respiratory illnesses, were swabbed, and tested positive for common cold Corona virus infections in 2019 (U. Chicago protocol # 09-043-A).

Historical controls and healthy controls: 30 samples from a US cohort was enrolled at the Rockefeller University Hospital in New York City in 2012 in accordance with a protocol approved by the Institutional Review Board of Rockefeller University (protocol #TWA-0804), in compliance with guidelines of the International Conference on Harmonization Good Clinical Practice guidelines, and was registered on www.clinicaltrials.gov (NCT01967238). Blood samples were drawn from healthy adult volunteers between the ages of 18-64. 50 samples were obtained from a Ugandan cohort of women and children enrolled in PROMOTE (NCT 02163447), a randomized clinical trial of novel antimalarial chemoprevention regimens in Eastern Uganda (Jagannathan, P. et al. PLoS Med 15, e1002606 (2018)). The study was approved by the Institutional Review Boards of the Makerere University School of Biomedical Sciences, the Uganda National Council for Science and Technology, and the University of California San Francisco. Written informed consent was obtained from all study participants. 50 samples were obtained from children under 18 years of age enrolled in a study of acute febrile illness in Nepal. The study was approved by the Nepal Health Research Council, Kathmandu University Institutional Review Board, and Stanford University Institutional Review Board (protocol #29992).

### IgG Fc Glycan and IgG Subclass Analysis

Methods for relative quantification of Fc Glycans and IgG subclasses have been previously described (Wang, T.T. et al., Cell 162, 160-169 (2015); and Wang, T.T. et al., Science 355, 395-398 (2017)). Briefly, IgGs were isolated from serum by protein G purification. Antigen-specific IgGs were isolated on NHS agarose resin (ThermoFisher; 26196) coupled to the protein of interest. Following tryptic digestion of purified IgG bound to antigen-coated beads, nanoLC-MS/MS analysis for characterization of glycosylation sites was performed on an UltiMate3000 nanoLC (Dionex) coupled with a hybrid triple quadrupole linear ion trap mass spectrometer, the 4000 Q Trap (SCIEX). MS data acquisition was performed using Analyst 1.6.1 software (SCIEX) for precursor ion scan triggered information dependent acquisition (IDA) analysis for initial discovery-based identification.

For quantitative analysis of the glycoforms at the N297 site of IgG1, multiple-reaction monitoring (MRM) analysis for selected target glycopeptide was applied using the nanoLC-4000 Q Trap platform to the samples which had been digested with trypsin. The m/z of 4-charged ions for all different glycoforms as Q1 and the fragment ion at m/z 366.1 as Q3 for each of transition pairs were used for MRM assays. A native IgGs tryptic peptide (131-GTLVTVSSASTK-142) (SEQ ID NO: 1) with transition pair of, 575.9⁺²/780.4 was used as a reference peptide for normalization. IgG subclass distribution was quantitatively determined by nanoLC-MRM analysis of tryptic peptides following removal of glycans from purified IgGs with PNGase F. Here the m/z value of fragment ions for monitoring transition pairs was always larger than that of their precursor ions to enhance the selectivity for unmodified targeted peptides and the reference peptide. All raw MRM data was processed using MultiQuant 2.1.1 (SCIEX). All MRM peak areas were automatically integrated and inspected manually. In the case where the automatic peak integration by MultiQuant failed, manual integration was performed using the MultiQuant software.

### Binding Affinity Measurements Using Biolayer Interferometry (BLI)

Total IgGs were purified from sera of 13 COVID-19 patients Protein G beads. The binding affinities of patient IgGs and high and low afucosylated 3022 mAbs were determined by biolayer interferometry (BLI) using an OctetQK instrument (Pall ForteBio). Human recombinant CD16a (Sino Biological) was captured onto the amine reactive second-generation (AR2G) biosensors using the amine reactive second-generation reagent kit (Pall ForteBio). The CD16a bound sensors were dipped into a concentration series (3.33 µM, 1.7 µM, 0.832 µM and 0.33 µM) of IgGs in PBST to determine the binding kinetics. An equal number of unliganded biosensors dipped into the analytes served as controls for referencing.

The traces were processed using ForteBio Data Analysis Software version 8.0.3.5 and corrected for non-specific binding. The data was fitted globally to a simple 1:1 Langmuir interaction model.

### NK Cell Degranulation Assay

PBMCs were isolated from whole blood collected from healthy blood donors post-plateletpheresis (Stanford Blood Center) using SepMate Isolation Tubes (STEMCELL). Cells were plated in a 96-well round-bottom plate (CELLSTAR) at a density of 3X10⁶ cells/mL of complete RPMI-1640 media supplemented with 1X penicillin-streptomycin-glutamine, 1 mM sodium pyruvate, and 1X MEM Non-Essential Amino Acids, 10% heat-inactivated fetal bovine serum (Gibco), and 1 ng/mL IL-15 (STEMCELL) and rested overnight at 37° C. in a 5% CO₂ incubator (Panasonic). The following morning, cell culture media was replaced with complete RPMI containing anti-CD107a antibody (BioLegend; clone H4A3). PBMCs were promptly stimulated for 6 hr at 37° C. with immune complexes formed by incubating purified patient IgG with SARS-CoV-2 receptor-binding domain protein at a molar ratio of 30:1 for 1 hr at room temperature. 1 hr into stimulation, culture media was supplemented with 1X Brefeldin A (BioLegend) for the remaining 5 hr of culture. Cells were then isolated, stained for cell viability using Live/Dead Fixable Staining Kit (Thermo Fisher) as well as CD3 (clone OKT3), CDllc (clone S-HCL-3), CD14 (clone 63D3), CD16 (clone 3G8), CD56 (clone HCD56), and HLA-DR (clone L243) surface markers (BioLegend). After staining, cells were fixed and acquired using an Attune NxT flow cytometer (Invitrogen). NK cells were defined as viable CD3⁻CD14⁻CD16⁺ CD56⁺ HLA-DR⁻ cells. NK cell degranulation was measured and reported as the fold change of NK cells positive for CD107a over control.

### Monocyte Stimulation and Cytokine Measurements

Monocytes were isolated from healthy donor blood (Stanford Blood Center) using RosetteSep Human Monocyte Enrichment Kit (STEMCELL) per manufacturer instructions. Monocytes were cultured at a density of 2×10⁶ cells/mL in RPMI 1640 media supplemented with 1X non-essential amino acids, sodium pyruvate, penicillin-streptomycin-glutamine (Gibco), and 10% fetal bovine serum (GE Healthcare Life Sciences).

Immune complexes were formed by incubating a dilution series of COVID-19 patient IgGs or anti-spike 3022 mAbs to SARS-CoV-2 spike-expressing delta-G-VSV pseudovirus for 1 hour at room temperature.

Monocytes were incubated with the various immune complexes or the pseudovirus only for 18 hours at 37° C. in a 5% CO₂ incubator. After 18 hours, cell-free supernatants were collected and proinflammatory cytokine concentrations were measured using a LEGENDplex bead array (BioLegend) per manufacturer instructions.

### Statistical Analysis

All data were analyzed with GraphPad Prism 8.0 software. Investigators were blinded to study subjects diagnoses during screening; COVID-19 patients and children were not known by investigators at the time of ELISA screening for RBD reactivity of serum or by investigators involved in relative quantitation of Fc glycoforms and IgG subclasses by mass spectrometry. R statistical package was used to generate the radar plots and perform multivariate linear regression analysis.

The various embodiments described above can be combined to provide further embodiments. All U.S. Patents, U.S. Patent Application Publications, U.S. Patent Application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of identifying a subject that is (a) symptomatic or prone to present one or more symptoms of COVID-19 and/or (b) at risk of progression to clinically significant COVID-19 infection or disease, sad method comprising
(i) obtaining a biological sample from a subject,
(ii) determining the amount of one or more of immunoglobulin fucosylation, galactosylation and/or bisection in the sample, and
(iii) comparing the fucosylation, galactosylation and/or bisection from a blood sample from a healthy adult donor;
wherein a reduced level of fucosylation, galactosylation and/or bisection when compared with the healthy adult donor is indicative of a subject that is symptomatic or prone to present one or more symptoms of COVID-19 and/or (b) prone to progress to severe COVID-19 disease.

2. The method of claim 1 wherein the immunoglobulin is IgG.

3. The method of claim 1 wherein the amount of fucosylation is determined.

4. The method of claim 3 wherein the amount of fucosylation is determined, wherein the level of afucosylated Fc glycans is defined as 5 percent or greater or 10 percent or greater.

5. The method of claim 1, wherein the subject is a human.

6. The method of claim 1, wherein the biological sample is blood or a blood fraction.

7. A method of treating a subject acutely infected with SARS-CoV-2 and at risk of progression to clinically significant COVID-19 infection or disease, the method comprising administering to the subject a therapeutic agent or vaccine following identification the subject is symptomatic or prone to present one or more symptoms of COVID-19 and/or (b) prone to progress to severe COVID-19 disease according to any one of claims 1-6.
